(19) United States
(12) Patent Application Publication
(10) Pub. No.: US 2020/0188699 A1
(43) Pub. Date: Jun. 18, 2020

(54) SYSTEMS AND METHODS FOR A WEARABLE DEVICE FOR ACOUSTIC STIMULATION

(71) Applicant: EpilepsyCo Inc., Guilford, CT (US)

(72) Inventors: Eric Kabrams, Redwood City, CA (US); Kamyar Firouzi, Palo Alto, CA (US)

(73) Assignee: EpilepsyCo Inc., Guilford, CT (US)

(21) Appl. No.: 16/714,585

(22) Filed: Dec. 13, 2019

Related U.S. Application Data
(60) Provisional application No. 62/822,709, filed on Mar. 22, 2019.
(60) Provisional application No. 62/822,697, filed on Mar. 22, 2019.
(60) Provisional application No. 62/822,684, filed on Mar. 22, 2019.
(60) Provisional application No. 62/822,679, filed on Mar. 22, 2019.
(60) Provisional application No. 62/822,675, filed on Mar. 22, 2019.
(60) Provisional application No. 62/822,668, filed on Mar. 22, 2019.
(60) Provisional application No. 62/822,657, filed on Mar. 22, 2019.
(60) Provisional application No. 62/779,188, filed on Dec. 13, 2018.

(51) Int. Cl. A61N 7/00

(52) U.S. Cl. CPC A61N 7/00; A61B 5/0476; A61N 2007/0026

(57) ABSTRACT

In some aspects, a device wearable by a person includes a transducer configured to apply to the brain of the person acoustic signals.

### CROSS REFERENCE TO RELATED APPLICATIONS

This application claims priority under 35 U.S.C. § 119(e) to U.S. Provisional Application Ser. No. 62/779,188, titled “NONINVASIVE NEUROLOGICAL DISORDER TREATMENT MODALITY,” filed Dec. 13, 2018, U.S. Provisional Application Ser. No. 62/822,709, titled “SYSTEMS AND METHODS FOR A WEARABLE DEVICE INCLUDING STIMULATION AND MONITORING COMPONENTS,” filed Mar. 22, 2019, U.S. Provisional Application Ser. No. 62/822,697, titled “SYSTEMS AND METHODS FOR A WEARABLE DEVICE FOR SUBSTANTIALLY NON-DESTRUCTIVE ACOUSTIC STIMULATION,” filed Mar. 22, 2019, U.S. Provisional Application Ser. No. 62/822,684, titled “SYSTEMS AND METHODS FOR A WEARABLE DEVICE FOR RANDOMIZED ACOUSTIC STIMULATION,” filed Mar. 22, 2019, U.S. Provisional Application Ser. No. 62/822,679, titled “SYSTEMS AND METHODS FOR A WEARABLE DEVICE FOR TREATING A NEUROLOGICAL DISORDER USING ULTRASOUND STIMULATION,” filed Mar. 22, 2019, U.S. Provisional Application Ser. No. 62/822,675, titled “SYSTEMS AND METHODS FOR A DEVICE FOR STEERING ACOUSTIC STIMULATION USING MACHINE LEARNING,” filed Mar. 22, 2019, U.S. Provisional Application Ser. No. 62/822,668, titled “SYSTEMS AND METHODS FOR A DEVICE USING A STATISTICAL MODEL TRAINED ON ANNOTATED SIGNAL DATA,” filed Mar. 22, 2019, and U.S. Provisional Application Ser. No. 62/822,657, titled “SYSTEMS AND METHODS FOR A DEVICE FOR ENERGY EFFICIENT MONITORING OF THE BRAIN,” filed Mar. 22, 2019, all of which are hereby incorporated herein by reference in their entireties.

### BACKGROUND

Recent estimates by the World Health Organization (WHO) have placed neurological disorders as constituting more than 6% of the global burden of disease. Such neurological disorders can include epilepsy, Alzheimer's disease, and Parkinson's disease.

For example, about 65 million people worldwide suffer from epilepsy. The United States itself has about 3.4 million people suffering from epilepsy with an estimated $15 billion economic impact. These patients suffer from symptoms such as recurrent seizures, which are episodes of excessive and synchronized neural activity in the brain. Because more than 70% of epilepsy patients live with suboptimal control of their seizures, such symptoms can be challenging for patients in school, in social and employment situations, in everyday activities like driving, and even in independent living.

### SUMMARY

In some aspects, a device wearable by or attached to or implanted within a person includes a sensor configured to detect a signal from the brain of the person and a transducer configured to apply to the brain an acoustic signal.

In some embodiments, the sensor includes an electroencephalogram (EEG) sensor, and the signal includes an EEG signal.

In some embodiments, the transducer includes an ultrasound transducer, and the acoustic signal includes an ultrasound signal.

In some embodiments, the ultrasound signal has a frequency between 100 kHz and 1 MHz, a spatial resolution between 0.001 cm³ and 0.1 cm³, and/or a power density between 1 and 100 watts/cm² as measured by spatial-peak pulse-average intensity.

In some embodiments, the ultrasound signal has a low power density, e.g., between 1 and 100 watts/cm², and is substantially non-destructive with respect to tissue when applied to the brain.

In some embodiments, the sensor and the transducer are disposed on the head of the person in a non-invasive manner.

In some embodiments, the device includes a processor in communication with the sensor and the transducer. The processor is programmed to receive, from the sensor, the signal detected from the brain and transmit an instruction to the transducer to apply to the brain the acoustic signal.

In some embodiments, the processor is programmed to transmit the instruction to the transducer to apply to the brain the acoustic signal at one or more random intervals.

In some embodiments, the device includes at least one other transducer configured to apply to the brain an acoustic signal, and the processor is programmed to select one of the transducers to transmit the instruction to apply to the brain the acoustic signal at the one or more random intervals.

In some embodiments, the processor is programmed to analyze the signal to determine whether the brain is exhibiting a symptom of a neurological disorder and transmit the instruction to the transducer to apply to the brain the acoustic signal in response to determining that the brain is exhibiting the symptom of the neurological disorder.

In some embodiments, the acoustic signal suppresses a symptom of a neurological disorder.

In some embodiments, the neurological disorder includes one or more of stroke, Parkinson's disease, migraine, tremors, frontotemporal dementia, traumatic brain injury, depression, anxiety, Alzheimer's disease, dementia, multiple sclerosis, schizophrenia, brain damage, neurodegeneration, central nervous system (CNS) disease, encephalopathy, Huntington's disease, autism, attention deficit hyperactivity disorder (ADHD), amyotrophic lateral sclerosis (ALS), and concussion.

In some embodiments, the symptom includes a seizure.

In some embodiments, the signal includes an electrical signal, a mechanical signal, an optical signal, and/or an infrared signal.

In some aspects, a method for operating a device wearable by or attached to or implanted within a person, the device including a sensor configured to detect a signal from the brain of the person and a transducer configured to apply to the brain an acoustic signal, includes receiving, from the sensor, the signal detected from the brain and applying to the brain, with the transducer, the acoustic signal.

In some aspects, an apparatus includes a device worn by or attached to or implanted within a person. The device includes a sensor configured to detect a signal from the brain of the person and a transducer configured to apply to the brain an acoustic signal.

In some aspects, a device wearable by a person includes a sensor configured to detect a signal from the brain of the person and a transducer configured to apply to the brain an ultrasound signal. The ultrasound signal has a low power density, e.g., between 1 and 100 watts/cm², and is substantially non-destructive with respect to tissue when applied to the brain.

In some embodiments, the sensor and the transducer are disposed on the head of the person in a non-invasive manner.

In some embodiments, the sensor includes an electroencephalogram (EEG) sensor, and the signal includes an EEG signal.

In some embodiments, the transducer includes an ultrasound transducer.

In some embodiments, the ultrasound signal has a frequency between 100 kHz and 1 MHz, a spatial resolution between 0.001 cm³ and 0.1 cm³, and/or the low power density between 1 and 100 watts/cm² as measured by spatial-peak pulse-average intensity.

In some embodiments, the ultrasound signal suppresses a symptom of a neurological disorder.

In some embodiments, the neurological disorder includes one or more of stroke, Parkinson's disease, migraine, tremors, frontotemporal dementia, traumatic brain injury, depression, anxiety, Alzheimer's disease, dementia, multiple sclerosis, schizophrenia, brain damage, neurodegeneration, central nervous system (CNS) disease, encephalopathy, Huntington's disease, autism, attention deficit hyperactivity disorder (ADHD), amyotrophic lateral sclerosis (ALS), and concussion.

In some embodiments, the symptom includes a seizure.

In some embodiments, the signal includes an electrical signal, a mechanical signal, an optical signal, and/or an infrared signal.

In some aspects, a method for operating a device wearable by a person, the device including a sensor configured to detect a signal from the brain of the person and a transducer configured to apply to the brain an ultrasound signal, includes applying to the brain the ultrasound signal. The ultrasound signal has a low power density, e.g., between 1 and 100 watts/cm², and is substantially non-destructive with respect to tissue when applied to the brain.

In some aspects, a method includes applying to the brain of a person, by a device worn by or attached to the person, an ultrasound signal.

In some aspects, an apparatus includes a device worn by or attached to a person. The device includes a sensor configured to detect a signal from the brain of the person and a transducer configured to apply to the brain an ultrasound signal. The ultrasound signal has a low power density, e.g., between 1 and 100 watts/cm², and is substantially non-destructive with respect to tissue when applied to the brain.

In some aspects, a device wearable by a person includes a transducer configured to apply to the brain of the person acoustic signals.

In some embodiments, the transducer is configured to apply to the brain of the person acoustic signals randomly.

In some embodiments, the transducer includes an ultrasound transducer, and the acoustic signals include an ultrasound signal.

In some embodiments, the ultrasound signal has a frequency between 100 kHz and 1 MHz, a spatial resolution between 0.001 cm³ and 0.1 cm³, and/or a power density between 1 and 100 watts/cm² as measured by spatial-peak pulse-average intensity.

In some embodiments, the ultrasound signal has a low power density, e.g., between 1 and 100 watts/cm², and is substantially non-destructive with respect to tissue when applied to the brain.

In some embodiments, the transducer is disposed on the head of the person in a non-invasive manner.

In some embodiments, the acoustic signal suppresses a symptom of a neurological disorder.

In some embodiments, the neurological disorder includes one or more of stroke, Parkinson's disease, migraine, tremors, frontotemporal dementia, traumatic brain injury, depression, anxiety, Alzheimer's disease, dementia, multiple sclerosis, schizophrenia, brain damage, neurodegeneration, central nervous system (CNS) disease, encephalopathy, Huntington's disease, autism, attention deficit hyperactivity disorder (ADHD), amyotrophic lateral sclerosis (ALS), and concussion.

In some embodiments, the symptom includes a seizure.

In some aspects, a method for operating a device wearable by a person, the device including a transducer, includes applying to the brain of the person acoustic signals.

In some aspects, an apparatus includes a device worn by or attached to a person. The device includes a transducer configured to apply to the brain of the person acoustic signals.

In some aspects, a device wearable by or attached to or implanted within a person includes a sensor configured to detect an electroencephalogram (EEG) signal from the brain of the person and a transducer configured to apply to the brain a low power, substantially non-destructive ultrasound signal.

In some embodiments, the ultrasound signal has a frequency between 100 kHz and 1 MHz, a spatial resolution between 0.001 cm³ and 0.1 cm³, and/or a power density between 1 and 100 watts/cm² as measured by spatial-peak pulse-average intensity.

In some embodiments, the sensor and the transducer are disposed on the head of the person in a non-invasive manner.

In some embodiments, the ultrasound signal suppresses an epileptic seizure.

In some embodiments, the device includes a processor in communication with the sensor and the transducer. The processor is programmed to receive, from the sensor, the EEG signal detected from the brain and transmit an instruction to the transducer to apply to the brain the ultrasound signal.

In some embodiments, the processor is programmed to transmit the instruction to the transducer to apply to the brain the ultrasound signal at one or more random intervals.

In some embodiments, the device includes at least one other transducer configured to apply to the brain an ultrasound signal, and the processor is programmed to select one of the transducers to transmit the instruction to apply to the brain the ultrasound signal at the one or more random intervals.

In some embodiments, the processor is programmed to analyze the EEG signal to determine whether the brain is exhibiting the epileptic seizure and transmit the instruction to the transducer to apply to the brain the ultrasound signal in response to determining that the brain is exhibiting the epileptic seizure.

In some aspects, a method for operating a device wearable by or attached to or implanted within a person, the device including a sensor configured to detect an electroencephalogram (EEG) signal from the brain of the person and a transducer configured to apply to the brain a low power, substantially non-destructive ultrasound signal, includes receiving, by the sensor, the EEG signal and applying to the brain, with the transducer, the ultrasound signal.

In some aspects, an apparatus includes a device worn by or attached to or implanted within a person. The device includes a sensor configured to detect an electroencephalogram (EEG) signal from the brain of the person and a transducer configured to apply to the brain a low power, substantially non-destructive ultrasound signal.

In some aspects, a device includes a sensor configured to detect a signal from the brain of the person and a plurality of transducers, each configured to apply to the brain an acoustic signal. One of the plurality of transducers is selected using a statistical model trained on data from prior signals detected from the brain.

In some embodiments, the device includes a processor in communication with the sensor and the plurality of transducers. The processor is programmed to provide data from a first signal detected from the brain as input to the trained statistical model to obtain an output indicating a first predicted strength of a symptom of a neurological disorder and, based on the first predicted strength of the symptom, select one of the plurality of transducers in a first direction to transmit a first instruction to apply a first acoustic signal.

In some embodiments, the processor is programmed to provide data from a second signal detected from the brain as input to the trained statistical model to obtain an output indicating a second predicted strength of the symptom of the neurological disorder, in response to the second predicted strength being less than the first predicted strength, select one of the plurality of transducers in the first direction to transmit a second instruction to apply a second acoustic signal, and, in response to the second predicted strength being greater than the first predicted strength, select one of the plurality of transducers in a direction opposite to or different from the first direction to transmit the second instruction to apply the second acoustic signal.

In some embodiments, the s -al model comprises a deep learning network.

In some embodiments, the deep learning network comprises a Deep Convolutional Neural Network (DCNN) for encoding the data onto an n-dimensional representation space and a Recurrent Neural Network (RNN) for computing a detection score by observing changes in the representation space through time. The detection score indicates a predicted strength of the symptom of the neurological disorder.

In some embodiments, data from the prior signals detected from the brain is accessed from an electronic health record of the person.

In some embodiments, the sensor includes an electroencephalogram (EEG) sensor, and the signal includes an EEG signal.

In some embodiments, the transducer includes an ultrasound transducer, and the acoustic signal includes an ultrasound signal.

In some embodiments, the ultrasound signal has a frequency between 100 kHz and 1 MHz, a spatial resolution between 0.001 cm³ and 0.1 cm³, and/or a power density between 1 and 100 watts/cm² as measured by spatial-peak pulse-average intensity.

In some embodiments, the ultrasound signal has a low power density, e.g., between 1 and 100 watts/cm², and is substantially non-destructive with respect to tissue when applied to the brain.

In some embodiments, the sensor and the transducer are disposed on the head of the person in a non-invasive manner.

In some embodiments, the acoustic signal suppresses a symptom of a neurological disorder.

In some embodiments, the neurological disorder includes one or more of stroke, Parkinson's disease, migraine, tremors, frontotemporal dementia, traumatic brain injury, depression, anxiety, Alzheimer's disease, dementia, multiple sclerosis, schizophrenia, brain damage, neurodegeneration, central nervous system (CNS) disease, encephalopathy, Huntington's disease, autism, attention deficit hyperactivity disorder (ADHD), amyotrophic lateral sclerosis (ALS), and concussion.

In some embodiments, the symptom includes a seizure.

In some embodiments, the signal includes an electrical signal, a mechanical signal, an optical signal, and/or an infrared signal.

In some aspects, a method for operating a device, the device including a sensor configured to detect a signal from the brain of the person and a plurality of transducers, each configured to apply to the brain an acoustic signal, includes selecting one of the plurality of transducers using a statistical model trained on data from prior signals detected from the brain.

In some aspects, an apparatus includes a device that includes a sensor configured to detect a signal from the brain of the person and a plurality of transducers, each configured to apply to the brain an acoustic signal. The device is configured to select one of the plurality of transducers using a statistical model trained on data from prior signals detected from the brain.

In some aspects, a device includes a sensor configured to detect a signal from the brain of the person and a plurality of transducers, each configured to apply to the brain an acoustic signal. One of the plurality of transducers is selected using a statistical model trained on signal data annotated with one or more values relating to identifying a health condition.

In some embodiments, the signal data annotated with the one or more values relating to identifying the health condition comprises the signal data annotated with respective values relating to increasing strength of a symptom of a neurological disorder.

In some embodiments, the statistical model was trained on data from prior signals detected from the brain annotated with the respective values between 0 and 1 relating to increasing strength of the symptom of the neurological disorder.

In some embodiments, the statistical model includes a loss function having a regularization term that is proportional to a variation of outputs of the statistical model, an L1/L2 norm of a derivative of the outputs, or an L1/L2 norm of a second derivative of the outputs.

In some embodiments, the device includes a processor in communication with the sensor and the plurality of transducers. The processor is programmed to provide data from a first signal detected from the brain as input to the trained statistical model to obtain an output indicating a first predicted strength of the symptom of the neurological disorder and, based on the first predicted strength of the symptom, select one of the plurality of transducers in a first direction to transmit a first instruction to apply a first acoustic signal.

In some embodiments, the processor is programmed to provide data from a second signal detected from the brain as input to the trained statistical model to obtain an output indicating a second predicted strength of the symptom of the neurological disorder, in response to the second predicted strength being less than the first predicted strength, select one of the plurality of transducers in the first direction to transmit a second instruction to apply a second acoustic signal, and, in response to the second predicted strength being greater than the first predicted strength, select one of the plurality of transducers in a direction opposite to or different from the first direction to transmit the second instruction to apply the second acoustic signal.

In some embodiments, the trained statistical model comprises a deep learning network.

In some embodiments, the deep learning network comprises a Deep Convolutional Neural Network (DCNN) for encoding the data onto an n-dimensional representation space and a Recurrent Neural Network (RNN) for computing a detection score by observing changes in the representation space through time. The detection score indicates a predicted strength of the symptom of the neurological disorder.

In some embodiments, the signal data includes data from prior signals detected from the brain that is accessed from an electronic health record of the person.

In some embodiments, the sensor includes an electroencephalogram (EEG) sensor, and the signal includes an EEG signal.

In some embodiments, the transducer includes an ultrasound transducer, and the acoustic signal includes an ultrasound signal.

In some embodiments, the ultrasound signal has a frequency between 100 kHz and 1 MHz, a spatial resolution between 0.001 cm³ and 0.1 cm³, and/or a power density between 1 and 100 watts/cm² as measured by spatial-peak pulse-average intensity.

In some embodiments, the ultrasound signal has a low power density, e.g., between 1 and 100 watts/cm², and is substantially non-destructive with respect to tissue when applied to the brain.

In some embodiments, the sensor and the transducer are disposed on the head of the person in a non-invasive manner.

In some embodiments, the acoustic signal suppresses the symptom of the neurological disorder.

In some embodiments, the neurological disorder includes one or more of stroke, Parkinson's disease, migraine, tremors, frontotemporal dementia, traumatic brain injury, depression, anxiety, Alzheimer's disease, dementia, multiple sclerosis, schizophrenia, brain damage, neurodegeneration, central nervous system (CNS) disease, encephalopathy, Huntington's disease, autism, attention deficit hyperactivity disorder (ADHD), amyotrophic lateral sclerosis (ALS), and concussion.

In some embodiments, the symptom includes a seizure. In some embodiments, the signal includes an electrical signal, a mechanical signal, an optical signal, and/or an infrared signal.

In some aspects, a method for operating a device, the device including a sensor configured to detect a signal from the brain of the person and a plurality of transducers, each configured to apply to the brain an acoustic signal, includes selecting one of the plurality of transducers using a statistical model trained on signal data annotated with one or more values relating to identifying a health condition.

In some aspects, an apparatus includes a device that includes a sensor configured to detect a signal from the brain of the person and a plurality of transducers, each configured to apply to the brain an acoustic signal. The device is configured to select one of the plurality of transducers using a statistical model trained on signal data annotated with one or more values relating to identifying a health condition.

In some aspects, a device includes a sensor configured to detect a signal from the brain of the person and a first processor in communication with the sensor. The first processor is programmed to identify a health condition and, based on the identified health condition, provide data from the signal to a second processor outside the device to corroborate or contradict the identified health condition.

In some embodiments, identifying the health condition comprises predicting a strength of a symptom of a neurological disorder.

In some embodiments, the processor is programmed to provide data from the signal detected from the brain as input to a first trained statistical model to obtain an output indicating the predicted strength, determine whether the predicted strength exceeds a threshold indicating presence of the symptom, and, in response to the predicted strength exceeding the threshold, transmit data from the signal to a second processor outside the device.

In some embodiments, the first statistical model was trained on data from prior signals detected from the brain.

In some embodiments, the first trained statistical model is trained to have high sensitivity and low specificity, and the first processor using the first trained statistical model uses a smaller amount of power than the first processor using the second trained statistical model.

In some embodiments, the second processor is programmed to provide data from the signal to a second trained statistical model to obtain an output to corroborate or contradict the predicted strength.

In some embodiments, the second trained statistical model is trained to have high sensitivity and high specificity.

In some embodiments, the first trained statistical model and/or the second trained statistical model comprise a deep learning network.

In some embodiments, the deep learning network comprises a Deep Convolutional Neural Network (DCNN) for encoding the data onto an n-dimensional representation space and a Recurrent Neural Network (RNN) for computing a detection score by observing changes in the representation space through time. The detection score indicates a predicted strength of the symptom of the neurological disorder.

In some embodiments, the sensor includes an electroencephalogram (EEG) sensor, and the signal includes an EEG signal.

In some embodiments, the sensor is disposed on the head of the person in a non-invasive manner.

In some embodiments, the neurological disorder includes one or more of stroke, Parkinson's disease, migraine, tremors, frontotemporal dementia, traumatic brain injury, depression, anxiety, Alzheimer's disease, dementia, multiple sclerosis, schizophrenia, brain damage, neurodegeneration, central nervous system (CNS) disease, encephalopathy, Huntington's disease, autism, attention deficit hyperactivity disorder (ADHD), amyotrophic lateral sclerosis (ALS), and concussion.

In some embodiments, the symptom includes a seizure.

In some embodiments, the signal includes an electrical signal, a mechanical signal, an optical signal, and/or an infrared signal.

In some aspects, a method for operating a device, the device including a sensor configured to detect a signal from the brain of the person and a transducer configured to apply to the brain an acoustic signal, includes identifying a health condition and, based on the identified health condition, providing data from the signal to a second processor outside the device to corroborate or contradict the identified health condition.

In some aspects, an apparatus includes a device that includes a sensor configured to detect a signal from the brain of the person and a transducer configured to apply to the brain an acoustic signal. The device is configured to identify a health condition and, based on the identified health condition, provide data from the signal to a second processor outside the device to corroborate or contradict the identified health condition.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

Various aspects and embodiments will be described with reference to the following figures. The figures are not necessarily drawn to scale.

FIG. 1 shows a device wearable by a person, e.g., for treating a symptom of a neurological disorder, in accordance with some embodiments of the technology described herein.

FIGS. 2A-2B show illustrative examples of a device wearable by a person for treating a symptom of a neurological disorder and mobile device(s) executing an application in communication with the device, in accordance with some embodiments of the technology described herein.

FIG. 3A shows an illustrative example of a mobile device and/or a cloud server in communication with a device wearable by a person for treating a symptom of a neurological disorder, in accordance with some embodiments of the technology described herein.

FIG. 3B shows a block diagram of a mobile device and/or a cloud server in communication with a device wearable by a person for treating a symptom of a neurological disorder, in accordance with some embodiments of the technology described herein.

FIG. 4 shows a block diagram for a wearable device including stimulation and monitoring components, in accordance with some embodiments of the technology described herein.

FIG. 5 shows a block diagram for a wearable device for substantially non-destructive acoustic stimulation, in accordance with some embodiments of the technology described herein.

FIG. 6 shows a block diagram for a wearable device for acoustic stimulation, e.g., randomized acoustic stimulation, in accordance with some embodiments of the technology described herein.

FIG. 7 shows a block diagram for a wearable device for treating a neurological disorder using ultrasound stimulation, in accordance with some embodiments of the technology described herein.

FIG. 8 shows a block diagram for a device to steer acoustic stimulation, in accordance with some embodiments of the technology described herein.

FIG. 9 shows a flow diagram for a device to steer acoustic stimulation, in accordance with some embodiments of the technology described herein.

FIG. 10 shows a block diagram for a device using a statistical model trained on annotated signal data, in accordance with some embodiments of the technology described herein.

FIG. 11A shows a flow diagram for a device using a statistical model trained on annotated signal data, in accordance with some embodiments of the technology described herein.

FIG. 11B shows a convolutional neural network that may be used to detect one or more symptoms of a neurological disorder, in accordance with some embodiments of the technology described herein.

FIG. 11C shows an exemplary interface including predictions from a deep learning network, in accordance with some embodiments of the technology described herein.

FIG. 12 shows a block diagram for a device for energy efficient monitoring of the brain, in accordance with some embodiments of the technology described herein.

FIG. 13 shows a flow diagram for a device for energy efficient monitoring of the brain, in accordance with some embodiments of the technology described herein.

FIG. 14 shows a block diagram of an illustrative computer system that may be used in implementing some embodiments of the technology described herein.

### DETAILED DESCRIPTION

Conventional treatment options for neurological disorders, such as epilepsy, present a tradeoff between invasiveness and effectiveness. For example, surgery may be effective in treating epileptic seizures for some patients, but the procedure is invasive. In another example, while antiepileptic drugs are non-invasive, they may not be effective for some patients. Some conventional approaches have used implanted brain simulation devices to provide electrical stimulation in an attempt to prevent and treat symptoms of neurological disorders, such as seizures. Other conventional approaches have used high-intensity lasers and high-intensity ultrasound (HIFU) to ablate brain tissue. These approaches can be highly invasive and often are only implemented following successful seizure focus localization, i.e., locating the focus of the seizure in the brain in order to perform ablation of the brain tissue or target electrical stimulation at that location. However, these approaches are based on the assumption that destruction or electrical stimulation of the brain tissue at the focus will stop the seizures. While this may be the case for some patients, it is not the case for other patients suffering from the same or similar neurological disorders. While some patients see a reduction in seizures after resection or ablation, there are many patients who see no benefit or exhibit even worse symptoms than prior to the treatment. For example, some patients having moderately severe seizures develop very severe seizures after surgery, while some patients develop entirely different types of seizures. Therefore conventional approaches can be highly invasive, difficult to implement correctly, and still only beneficial to some patients.

The inventors have discovered an effective treatment option for neurological disorders that also is non-invasive or minimally-invasive and/or substantially non-destructive. The inventors have proposed the described systems and methods where, instead of trying to kill brain tissue in a one-time operation, the brain tissue is activated using acoustic signals, e.g., low-intensity ultrasound, delivered transcranially to stimulate neurons in certain brain regions in a substantially non-destructive manner. In some embodiments, the brain tissue may be activated at random intervals, e.g., sporadically throughout the day and/or night, thereby preventing the brain from settling into a seizure state. In some embodiments, the brain tissue may be activated in response to detecting that the patient's brain is exhibiting signs of a seizure, e.g., by monitoring electroencephalogram (EEG) measurements from the brain. Accordingly, some embodiments of the described systems and methods provide for non-invasive and/or substantially non-destructive treatment of symptoms of neurological disorders, such as stroke, Parkinson's, migraine, tremors, frontotemporal dementia, traumatic brain injury, depression, anxiety, Alzheimer's, dementia, multiple sclerosis, schizophrenia, brain damage, neurodegeneration, central nervous system (CNS) disease, encephalopathy, Huntington's, autism, ADHD, ALS, concussion, and/or other suitable neurological disorders.

For example, some embodiments of the described systems and methods may provide for treatment that allows one or more sensors to be placed on the scalp of the person. Therefore the treatment may be non-invasive because no surgery is required to dispose the sensors on the scalp for monitoring the brain of the person. In another example, some embodiments of the described systems and methods may provide for treatment that allows one or more sensors to be placed just below the scalp of the person. Therefore the treatment may be minimally-invasive because a subcutaneous surgery, or a similar procedure requiring small or no incisions, may be used to dispose the sensors just below the scalp for monitoring the brain of the person. In another example, some embodiments of the described systems and methods may provide for treatment that applies to the brain, with one or more transducers, a low-intensity ultrasound signal. Therefore the treatment may be substantially non-destructive because no brain tissue is ablated or resected during application of the treatment to the brain.

In some embodiments, the described systems and methods provide for a device wearable by a person in order to treat a symptom of a neurological disorder. The device may include a transducer that is configured to apply to the brain an acoustic signal. In some embodiments, the acoustic signal may be an ultrasound signal that is applied using a low spatial resolution, e.g., on the order of hundreds of cubic millimeters. Unlike conventional ultrasound treatment (e.g., HIFU) which is used for tissue ablation, some embodiments of the described systems and methods use lower spatial resolution for the ultrasound stimulation. The low spatial resolution requirements may reduce the stimulation frequency (e.g., on the order of 100 kHz-1 MHz), thereby allowing the system to operate at low energy levels as these lower frequency signals experience significantly lower attenuation when passing through the person's skull. This decrease in power usage may be suitable for substantially non-destructive use and/or for use in a wearable device. Accordingly, the low energy usage may enable some embodiments of the described systems and methods to be implemented in a device that is low power, always-on, and/or wearable by a person.

In some embodiments, the described systems and methods provide for a device wearable by a person that includes monitoring and stimulation components. The device may include a sensor that is configured to detect a signal, e.g., an electrical signal, a mechanical signal, an optical signal, an infrared signal, or another suitable type of signal, from the brain of the person. For example, the device may include an EEG sensor, or another suitable sensor, that is configured to detect an electrical signal such as an EEG signal, or another suitable signal, from the brain of the person. The device may include a transducer that is configured to apply to the brain an acoustic signal. For example, the device may include an ultrasound transducer that is configured to apply to the brain an ultrasound signal. In another example, the device may include a wedge transducer to apply to the brain an ultrasound signal. U.S. Patent. Application Publication No. 2018/0280735 provides further information on exemplary embodiments of wedge transducers. the entirety of which is incorporated by reference herein.

In some embodiments, the wearable device may include a processor in communication with the sensor and/or the transducer. The processor may receive, from the sensor, a signal detected from the brain. The processor may transmit an instruction to the transducer to apply to the brain the acoustic signal. In some embodiments, the processor may be programmed to analyze the signal to determine whether the brain is exhibiting a symptom of a neurological disorder, e.g., a seizure. The processor may be programmed to transmit the instruction to the transducer to apply to the brain the acoustic signal, e.g., in response to determining that the brain is exhibiting the symptom of the neurological disorder. The acoustic signal may suppress the symptom of the neurological disorder, e.g., a seizure.

In some embodiments, the ultrasound signal may have a low power density and be substantially non-destructive with respect to tissue when applied to the brain.

In some embodiments, the ultrasound transducer may be driven by a voltage waveform such that the power density, as measured by spatial-peak pulse-average intensity, of the acoustic focus of the ultrasound signal, characterized in water, is in the range of 1 to 100 watts/cm². When in use, the power density reaching the focus in the patient's brain may be attenuated by the patient's skull from the range described above by 1-20 dB. In some embodiments, the power density may be measured by the spatial-peak temporal average (Ispta) or another suitable metric. In some embodiments, a mechanical index, which measures at least a portion of the ultrasound signal's bioeffects, at the acoustic focus of the ultrasound signal may be determined. The mechanical index may be less than 1.9 to avoid cavitation at or near the acoustic focus.

In some embodiments, the ultrasound signal may have a frequency between 100 kHz and 1 MHz, or another suitable range. In some embodiments, the ultrasound signal may have a spatial resolution between 0.001 cm³ and 0.1 cm³, or another suitable range.

In some embodiments, the device may apply to the brain with the transducer an acoustic signal at one or more random intervals. For example, the device may apply to a patient's brain the acoustic signal at random times throughout the day and/or night, e.g., around every 10 minutes. In another example, for patients with generalized epilepsy, the device may stimulate the thalamus at random times throughout the day and/or night, e.g., around every 10 minutes. In some embodiments, the device may include another transducer. The device may select one of the transducers to apply to the brain the acoustic signal at one or more random intervals. In some embodiments, the device may include an array of transducers that can be programmed to aim an ultrasonic beam at any location within the skull or to create a pattern of ultrasonic radiation within the skull with multiple foci.

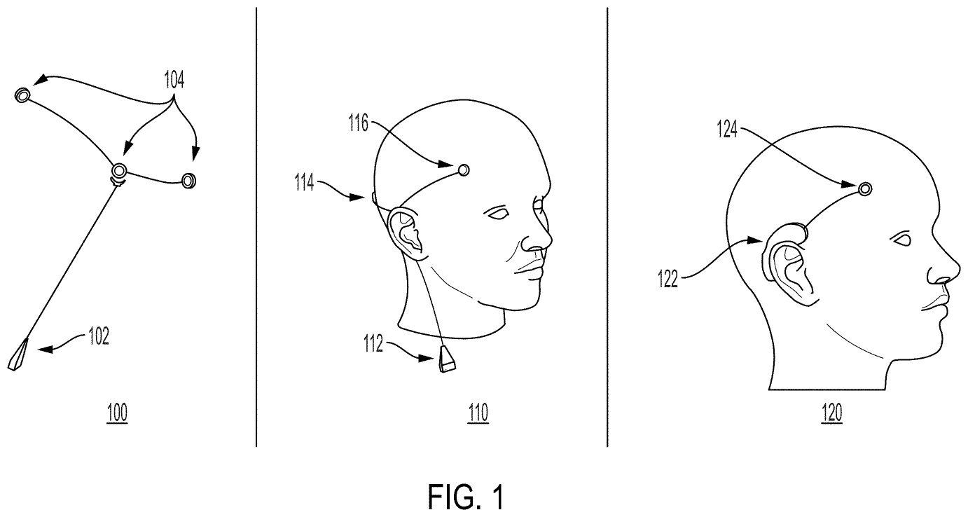

In some embodiments, the sensor and the transducer are disposed on the head of the person in a non-invasive manner. For example, the device may be disposed on the head of the person in a non-invasive manner, such as placed on the scalp of the person or in another suitable manner. An illustrative example of the device is described with respect to FIG. 1 below. In some embodiments, the sensor and the transducer are disposed on the head of the person in a minimally-invasive manner. For example, the device may be disposed on the head of the person through a subcutaneous surgery, or a similar procedure requiring small or no incisions, such as placed just below the scalp of the person or in another suitable manner.

In some embodiments, a seizure may be considered to occur when a large number of neurons fire synchronously with structured phase relationships. The collective activity of a population of neurons may be mathematically represented as a point evolving in a high-dimensional space, with each dimension corresponding to the membrane voltage of a single neuron. In this space, a seizure may be represented by a stable limit cycle, an isolated, periodic attractor. As the brain performs its daily tasks, its state, represented by a point in the high-dimensional space, may move around the space, tracing complicated trajectories. However, if this point gets too close to a certain dangerous region of space, e.g., the basin of attraction of the seizure, the point may get pulled into the seizure state. Depending on the patient, certain activities, such as sleep deprivation, alcohol consumption, and eating certain foods may have a propensity to push the brain state closer to the danger zone of the seizure's basin of attraction. Conventional treatment involving resecting/ablating the estimated source brain tissue of the seizure attempts to change the landscape in this space. While for some patients the seizure limit cycle may be removed, for others the old limit cycle may be become more strongly attracting or perhaps a new one may appear. Moreover, any type of surgery to brain tissue, including surgical placement of electrodes, is highly invasive, and because the brain is an incredibly large, complicated network, it may be non-trivial to predict the network-level effects of removing or otherwise impairing a spatially localized piece of brain tissue.

Some embodiments of the described systems and methods, rather than localizing the seizure and removing the estimated source brain tissue, monitor the brain using, e.g., EEG signals, to determine when the brain state is getting close to the basin of attraction for a seizure. Whenever it is detected that the brain state is getting close to this danger zone, the brain is perturbed using, e.g., an acoustic signal, to push the brain state out of the danger zone. In other words, rather than trying to change the landscape in this space, some embodiments of the described systems and methods learn what the landscape of the brain, monitor the brain state, and ping the brain when needed, thereby removing it from the danger zone. Some embodiments of the described systems and methods provide for non-invasive, substantially non-destructive neural stimulation, lower power dissipation (e.g., than other transcranial ultrasound therapies), and/or a suppression strategy coupled with a non-invasive electrical recording device.

For example, for patients with generalized epilepsy, some embodiments of the described systems and methods may stimulate the thalamus or another suitable region of the brain at random times throughout the day and/or night, e.g., around every 10 minutes. The device may use an ultrasound frequency of around 100 kHz-1 MHz at a power usage of around 1-100 watts/cm² as measured by spatial-peak pulse-average intensity. In another example, for patients with left temporal lobe epilepsy, some embodiments of the described systems and methods may stimulate the left temporal lobe or another suitable region of the brain in response to detecting an increased seizure risk level based on EEG signals (e.g., above some predetermined threshold). The left temporal lobe may be stimulated until the EEG signals indicate that the seizure risk level has decreased and/or until some maximum stimulation time threshold (e.g., several minutes) has been reached. The predetermined threshold may be determined using machine learning training algorithms trained on the patient's EEG recordings and a monitoring algorithm may measure the seizure risk level using the EEG signals.

In some embodiments, seizure suppression strategies can be categorized by their spatial and temporal resolution and can vary per patient. Spatial resolution refers to the size of the brain structures that are being activated/inhibited. In some embodiments, low spatial resolution may be a few hundred cubic millimeters, e.g., on the order of 0.1 cubic centimeters. In some embodiments, medium spatial resolution may be on the order of 0.01 cubic centimeters. In some embodiments, high spatial resolution may be a few cubic millimeters, e.g., on the order of 0.001 cubic centimeters. Temporal resolution generally refers to responsiveness of the stimulation. In some embodiments, low temporal resolution may include random stimulation with no regard for when seizures are likely to occur. In some embodiments, medium temporal resolution may include stimulation in response to a small increase in seizure probability. In some embodiments, high temporal resolution may include stimulation in response to detecting a high seizure probability, e.g., right after a seizure started. In some embodiments, using strategies with medium and high temporal resolution may require using a brain-activity recording device and running machine learning algorithms to detect the likelihood of a seizure occurring in the near future.

In some embodiments, the device may use a strategy with low-medium spatial resolution and low temporal resolution. The device may coarsely stimulate centrally connected brain structures to prevent seizures from occurring, using low power transcranial ultrasound. For example, the device may stimulate one or more regions of the brain with ultrasound stimulation of a low spatial resolution (e.g., on the order of hundreds of cubic millimeters) at random times throughout the day and/or night. The effect of such random stimulation may be to prevent the brain from settling into its familiar patterns that often lead to seizures. The device may target individual subthalamic nuclei and other suitable brain regions with high connectivity to prevent seizures from occurring.

In some embodiments, the device may employ a strategy with low-medium spatial resolution and medium-high temporal resolution. The device may include one or more sensors to non-invasively monitor the brain and detect a high level of seizure risk (e.g., higher probability that a seizure will occur within the hour). In response to detecting a high seizure risk level, the device may apply low power ultrasound stimulation that is transmitted through the skull, to the brain, activating and/or inhibiting brain structures to prevent/stop seizures from occurring. For example, the ultrasound stimulation may include frequencies from 100 kHz to 1 MHz and/or power density from 1 to 100 watts/cm² as measured by spatial-peak pulse-average intensity. The device may target brain structures such as the thalamus, piriform cortex, coarse-scale structures in the same hemisphere as seizure foci (e.g., for patients with localized epilepsy), and other suitable brain structures to prevent seizures from occurring.

FIG. 1 shows different aspects 100, 110, and 120 of a device wearable by a person for treating a symptom of a neurological disorder, in accordance with some embodiments of the technology described herein. The device may be a non-invasive seizure prediction and/or detection device. In some embodiments, in aspect 100, the device may include a local processing device 102 and one or more electrodes 104. The local processing device 102 may include a wristwatch, an arm band, a necklace, a wireless earbud, or another suitable device. The local processing device 102 may include a radio and/or a physical connector for transmitting data to a cloud server, a mobile phone, or another suitable device. The local processing device 102 may receive, from a sensor, a signal detected from the brain and transmit an instruction to a transducer to apply to the brain an acoustic signal. The electrodes 104 may include one or more sensors configured to detect a signal from the brain of the person, e.g., an EEG signal, and/or one or more transducers configured to apply to the brain an acoustic signal, e.g., an ultrasound signal. The acoustic signal may have a low power density and be substantially non-destructive with respect to tissue when applied to the brain. In some embodiments, one electrode may include either a sensor or a transducer. In some embodiments, one electrode may include both a sensor and a transducer. In some embodiments, one, 10, 20, or another suitable number of electrodes may be available. The electrodes may be removably attached to the device.

In some embodiments, in aspect 110, the device may include a local processing device 112, a sensor 114, and a transducer 116. The device may be disposed on the head of the person in a non-invasive manner, such as placed on the scalp of the person or in another suitable manner. The local processing device 112 may include a wristwatch, an arm band, a necklace, a wireless earbud, or another suitable device. The local processing device 112 may include a radio and/or a physical connector for transmitting data to a cloud server, a mobile phone, or another suitable device. The local processing device 112 may receive, from the sensor 114, a signal detected from the brain and transmit an instruction to the transducer 116 to apply to the brain an acoustic signal. The sensor 114 may be configured to detect a signal from the brain of the person, e.g., an EEG signal. The transducer 116 may be configured to apply to the brain an acoustic signal, e.g., an ultrasound signal. The acoustic signal may have a low power density and be substantially non-destructive with respect to tissue when applied to the brain. In some embodiments, one electrode may include either a sensor or a transducer. In some embodiments, one electrode may include both a sensor and a transducer. In some embodiments, one, 10, 20, or another suitable number of electrodes may be available. The electrodes may be removably attached to the device.

In some embodiments, in aspect 120, the device may include a local processing device 122 and an electrode 124. The device may be disposed on the head of the person in a non-invasive manner, such as placed over the ear of the person or in another suitable manner. The local processing device 122 may include a wristwatch, an arm band, a necklace, a wireless earbud, or another suitable device. The local processing device 122 may include a radio and/or a physical connector for transmitting data to a cloud server, a mobile phone, or another suitable device. The local processing device 122 may receive, from the electrode 124, a signal detected from the brain and/or transmit an instruction to the electrode 124 to apply to the brain an acoustic signal. The electrode 124 may include a sensor configured to detect a signal from the brain of the person, e.g., an EEG signal, and/or a transducer configured to apply to the brain an acoustic signal, e.g., an ultrasound signal. The acoustic signal may have a low power density and be substantially non-destructive with respect to tissue when applied to the brain. In some embodiments, the electrode 124 may include either a sensor or a transducer. In some embodiments, the electrode 124 may include both a sensor and a transducer. In some embodiments, one, 10, 20, or another suitable number of electrodes may be available. The electrodes may be removably attached to the device.

In some embodiments, the device may include one or more sensors for detecting sound, motion, optical signals, heart rate, and other suitable sensing modalities. For example, the sensor may detect an electrical signal, a mechanical signal, an optical signal, an infrared signal, or another suitable type of signal. In some embodiments, the device may include a wireless earbud, a sensor embedded in the wireless earbud, and a transducer. The sensor may detect a signal, e.g., an EEG signal, from the brain of the person while the wireless earbud is present in the person's ear. The wireless earbud may have an associated case or enclosure that includes a local processing device for receiving and processing the signal from the sensor and/or transmitting an instruction to the transducer to apply to the brain an acoustic signal.

In some embodiments, the device may include a sensor for detecting a mechanical signal, such as a signal with a frequency in the audible range. For example, the sensor may be used to detect an audible signal from the brain indicating a seizure. The sensor may be an acoustic receiver disposed on the scalp of the person to detect an audible signal from the brain indicating a seizure. In another example, the sensor may be an accelerometer disposed on the scalp of the person to detect an audible signal from the brain indicating a seizure. In this manner, the device may be used to “hear” the seizure around the time it occurs.

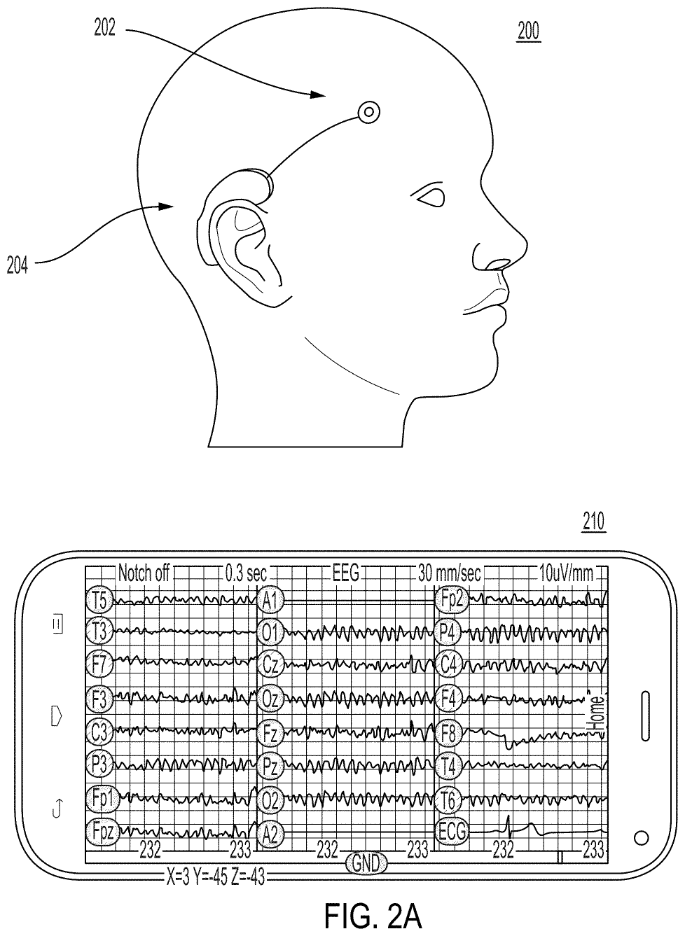

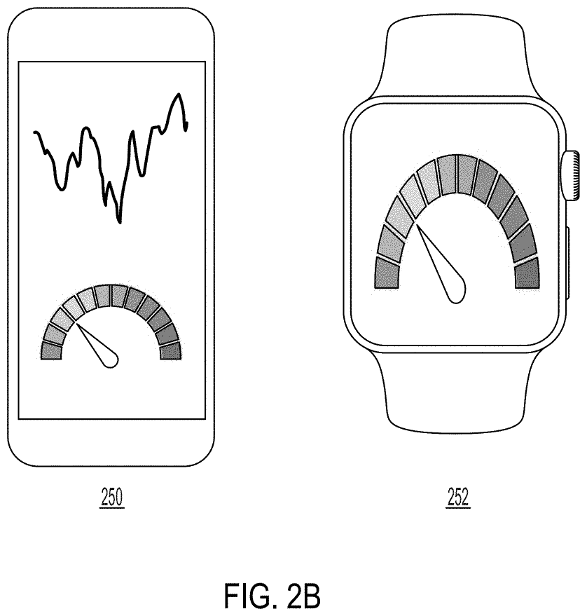

FIGS. 2A-2B show illustrative examples of a device wearable by a person for treating a symptom of a neurological disorder and mobile device(s) executing an application in communication with the device, in accordance with some embodiments of the technology described herein. FIG. 2A shows an illustrative example of a device 200 wearable by a person for treating a symptom of a neurological disorder and a mobile device 210 executing an application in communication with the device 200. In some embodiments, the device 200 may be capable of predicting seizures, detecting seizures and alerting users or caretakers, tracking and managing the condition, and/or suppressing symptoms of neurological disorders, such as seizures. The device 200 may connect to the mobile device 210, such as a mobile phone, watch, or another suitable device via BLUETOOTH, WIFI, or another suitable connection. The device 200 may monitor neuronal activity with one or more sensors 202 and share data with a user, a caretaker, or another suitable entity using processor 204. The device 200 may learn about individual patient patterns. The device 200 may access data from prior signals detected from the brain from an electronic health record of the person wearing the device 200.

FIG. 2B shows illustrative examples of mobile devices 250 and 252 executing an application in communication with a device wearable by a person for treating a symptom of a neurological disorder, e.g., device 200. For example, the mobile device 250 or 252 may display real-time seizure risk for the person suffering from the neurological disorder. In the event of a seizure, the mobile device 250 or 252 may alert the person, a caregiver, or another suitable entity. For example, the mobile device 250 or 252 may inform a caretaker that a seizure is predicted in the next 30 minutes, next hour, or another suitable time period. In another example, the mobile device 250 or 252 may send alerts to the caretaker when a seizure does occur and/or record seizure activity, such as signals from the brain, for the caretaker to refine treatment of the person's neurological disorder. In some embodiments, the wearable device 200 and/or the mobile device 250 or 252 may analyze a signal, such as an EEG signal, detected from the brain to determine whether the brain is exhibiting a symptom of a neurological disorder. The wearable device 200 may apply to the brain an acoustic signal, such as an ultrasound signal, in response to determining that the brain is exhibiting the symptom of the neurological disorder.

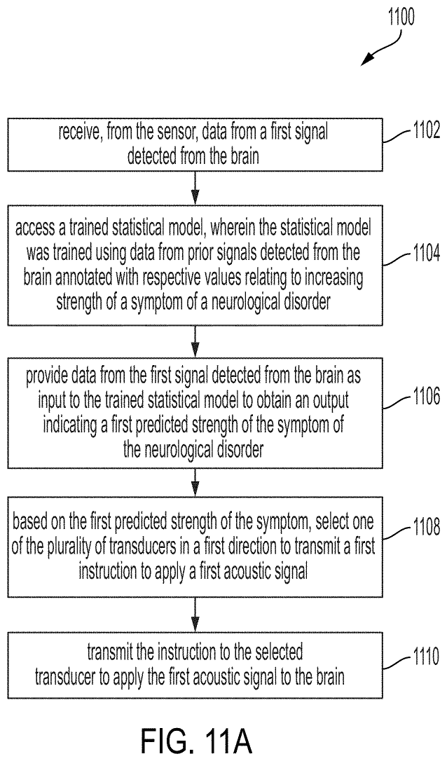

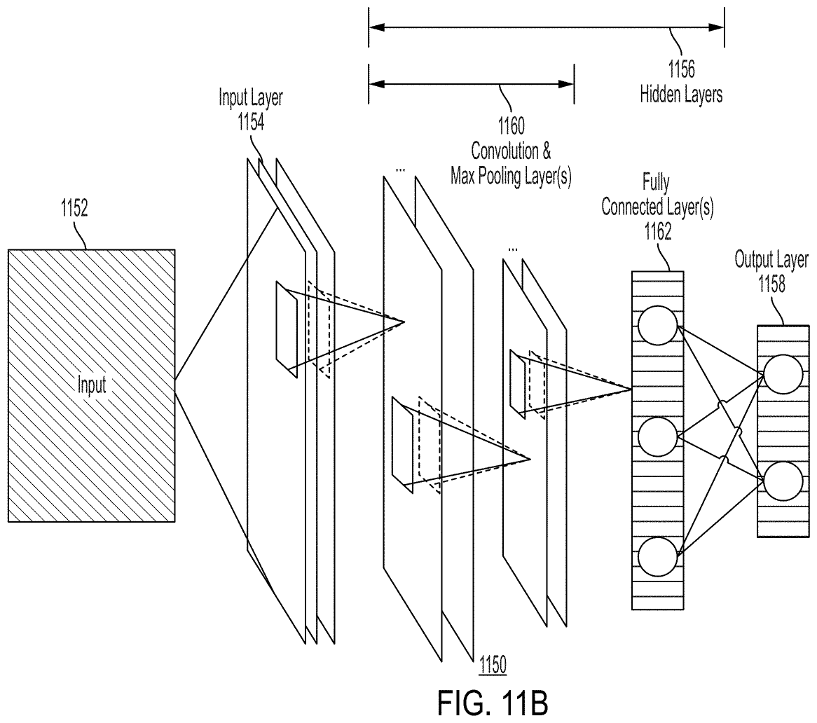

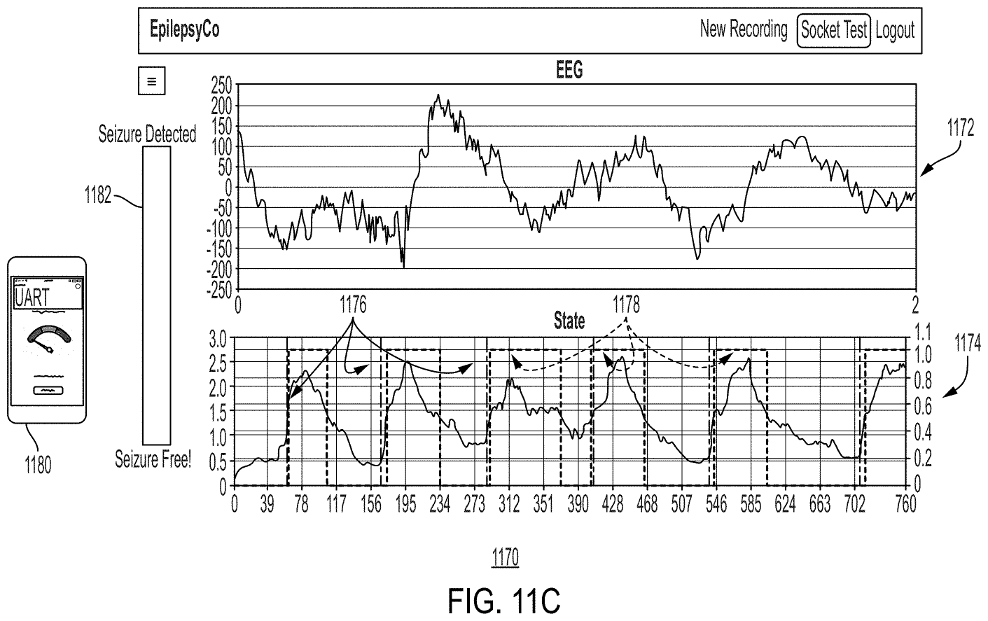

In some embodiments, the wearable device 200, the mobile device 250 or 252, and/or another suitable computing device may provide one or more signals, e.g., an EEG signal or another suitable signal, detected from the brain to a deep learning network to determine whether the brain is exhibiting a symptom of a neurological disorder, e.g., a seizure or another suitable symptom. The deep learning network may be trained on data gathered from a population of patients and/or the person wearing the wearable device 200. The mobile device 250 or 252 may generate an interface to warn the person and/or a caretaker when the person is likely to have a seizure and/or when the person will be seizure-free. In some embodiments, the wearable device 200 and/or the mobile device 250 or 252 may allow for two-way communication to and from the person suffering from the neurological disorder. For example, the person may inform the wearable device 200 via text, speech, or another suitable input mode that “I just had a beer, and I'm worried I may be more likely to have a seizure.” The wearable device 200 may respond using a suitable output mode that “Okay, the device will be on high alert.” The deep learning network may use this information to assist in future predictions for the person. For example, the deep learning network may add this information to data used for updating/training the deep learning network. In another example, the deep learning network may use this information as input to help predict the next symptom for the person. Additionally or alternatively, the wearable device 200 may assist the person and/or the caretaker in tracking sleep and/or diet patterns of the person suffering from the neurological disorder and provide this information when requested. The deep learning network may add this information to data used for updating/training the deep learning network and/or use this information as input to help predict the next symptom for the person. Further information regarding the deep learning network is provided with respect to FIGS. 11B and 11C.

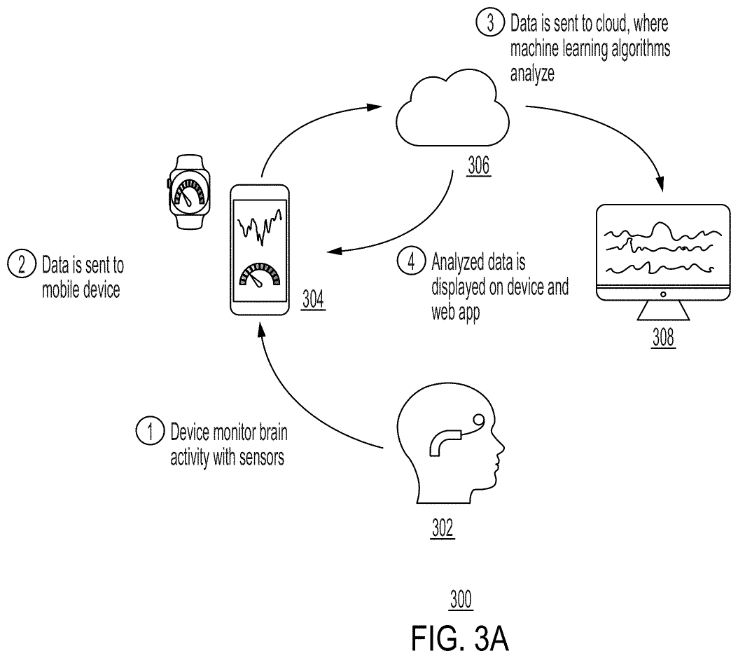

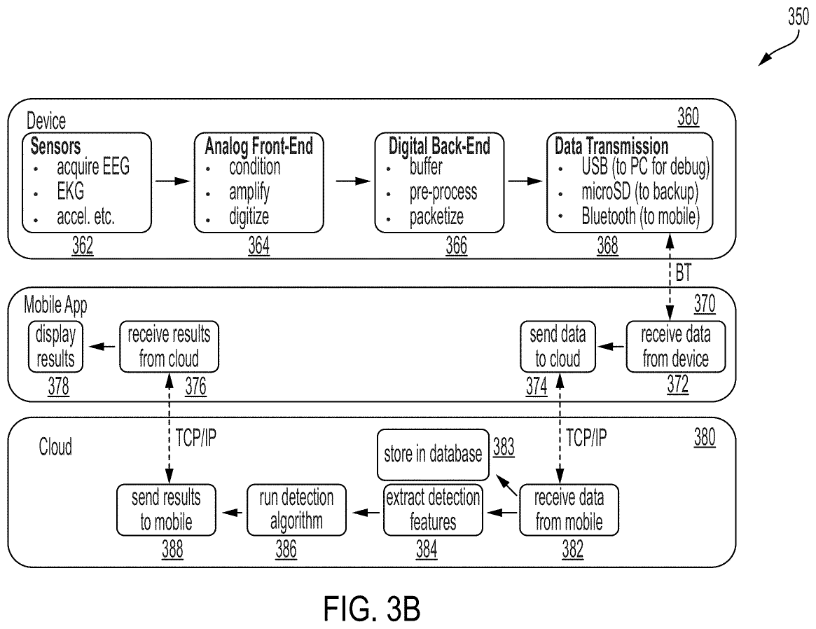

FIG. 3A shows an illustrative example 300 of a mobile device and/or a cloud server in communication with a device wearable by a person for treating a symptom of a neurological disorder, in accordance with some embodiments of the technology described herein. In this example, the wearable device 302 may monitor brain activity with one or more sensors and send the data to the person's mobile device 304, e.g., a mobile phone, a wristwatch, or another suitable mobile device. The mobile device 304 may analyze the data and/or send the data to a server 306, e.g., a cloud server. The server 306 may execute one or more machine learning algorithms to analyze the data. For example, the server 306 may use a deep learning network that takes the data or a portion of the data as input and generates output with information about one or more predicted symptoms, e.g., a predicted strength of a seizure. The analyzed data may be displayed on the mobile device 304 and/or an application on a computing device 308. For example, the mobile device 304 and/or computing device 308 may display real-time seizure risk for the person suffering from the neurological disorder. In the event of a seizure, the mobile device 304 and/or computing device 308 may alert the person, a caregiver, or another suitable entity. For example, the mobile device 304 and/or computing device 308 may inform a caretaker that a seizure is predicted in the next 30 minutes, next hour, or another suitable time period. In another example, the mobile device 304 and/or computing device 308 may send alerts to the caretaker when a seizure does occur and/or record seizure activity, such as signals from the brain, for the caretaker to refine treatment of the person's neurological disorder.

In some embodiments, one or more alerts may be generated by a machine learning algorithm trained to detect and/or predict seizures. For example, the machine learning algorithm may include a deep learning network, e.g., as described with respect to FIGS. 11B and 11C. When the algorithm detects that a seizure is present, or predicts that a seizure is likely to develop in the near future (e.g., within an hour), an alert may be sent to a mobile application. The interface of the mobile application may include bi-directional communication, e.g., in addition to the mobile application sending notifications to the patient, the patient may have the ability to enter information into the mobile application to improve the performance of the algorithm. For example, if the machine learning algorithm is not certain within a confidence threshold that the patient is having a seizure, it may send a question to the patient through the mobile application, asking the patient whether or not he/she recently had a seizure. If the patient answers no, the algorithm may take this into account and train or re-train accordingly.

FIG. 3B shows a block diagram 350 of a mobile device and/or a cloud server in communication with a device wearable by a person for treating a symptom of a neurological disorder, in accordance with some embodiments of the technology described herein. Device 360 may include a wristwatch, an arm band, a necklace, a wireless earbud, or another suitable device. The device 360 may include one or more sensors (block 362) to acquire signals from the brain (e.g., from EEG sensors, accelerometers, electrocardiogram (EKG) sensors, and/or other suitable sensors). The device 360 may include an analog front-end (block 364) for conditioning, amplifying, and/or digitizing the signals acquired by the sensors (block 362). The device 360 may include a digital back-end (block 366) for buffering, pre-processing, and/or packetizing the output signals from the analog front-end (block 364). The device 360 may include data transmission circuitry (block 368) for transmitting the data from the digital back-end (block 366) to a mobile application 370, e.g., via BLUETOOTH. Additionally or alternatively, the data transmission circuitry (block 368) may send debugging information to a computer, e.g., via USB, and/or send backup information to local storage, e.g., a microSD card.

The mobile application 370 may execute on a mobile phone or another suitable device. The mobile application 370 may receive data from the device 370 (block 372) and send the data to a cloud server 380 (block 374). The cloud server 380 may receive data from the mobile application 370 (block 382) and store the data in a database (block 383). The cloud server 380 may extract detection features (block 384), run a detection algorithm (block 386), and send results back to the mobile application 370 (block 388). Further details regarding the detection algorithm are described later in this disclosure, including with respect to FIGS. 11B and 11C. The mobile application 370 may receive the results from the cloud server 380 (block 376) and display the results to the user (block 378).

In some embodiments, the device 360 may transmit the data directly to the cloud server 380, e.g., via the Internet. The cloud server 380 may send the results to the mobile application 370 for display to the user. In some embodiments, the device 360 may transmit the data directly to the cloud server 380, e.g., via the Internet. The cloud server 380 may send the results back to the device 360 for display to the user. For example, the device 360 may be a wristwatch with a screen for displaying the results. In some embodiments, the device 360 may transmit the data to the mobile application 370, and the mobile application 370 may extract detection features, run a detection algorithm, and/or display the results to the user on the mobile application 370 and/or the device 360. Other suitable variations of interactions between the device 360, the mobile application 370, and/or the cloud server 380 may be possible and are within the scope of this disclosure.

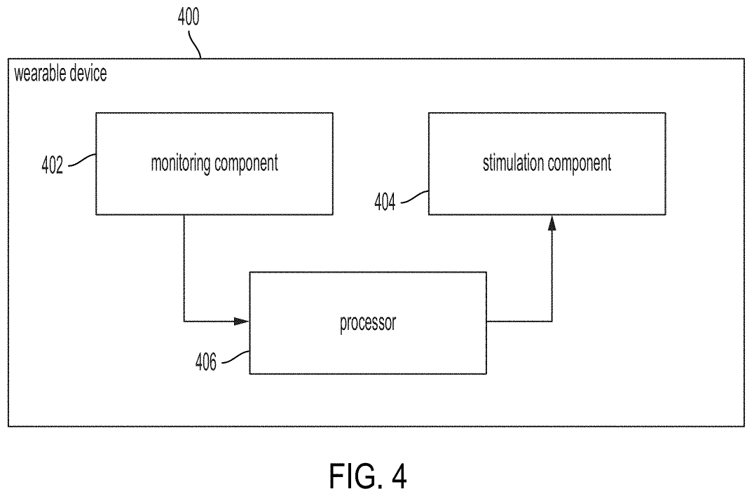

FIG. 4 shows a block diagram for a wearable device 400 including stimulation and monitoring components, in accordance with some embodiments of the technology described herein. The device 400 is wearable by (or attached to or implanted within) a person and includes a monitoring component 402, a stimulation component 404, and a processor 406. The monitoring component 402 may include a sensor that is configured to detect a signal, e.g., an electrical signal, a mechanical signal, an optical signal, an infrared signal, or another suitable type of signal, from the brain of the person. For example, the sensor may be an electroencephalogram (EEG) sensor, and the signal may be an electrical signal, such as an EEG signal. The stimulation component 404 may include a transducer configured to apply to the brain an acoustic signal. For example, the transducer may be an ultrasound transducer, and the acoustic signal may be an ultrasound signal. In some embodiments, the ultrasound signal may have a low power density and be substantially non-destructive with respect to tissue when applied to the brain. In some embodiments, the sensor and the transducer may be disposed on the head of the person in a non-invasive manner.

The processor 406 may be in communication with the monitoring component 402 and the stimulation component 404. The processor 406 may be programmed to receive, from the monitoring component 402, the signal detected from the brain and transmit an instruction to the stimulation component 404 to apply to the brain the acoustic signal. In some embodiments, the processor 406 may be programmed to transmit the instruction to the stimulation component 404 to apply to the brain the acoustic signal at one or more random intervals. In some embodiments, the stimulation component 404 may include two or more transducers, and the processor 406 may be programmed to select one of the transducers to transmit the instruction to apply to the brain the acoustic signal at one or more random intervals.

In some embodiments, the processor 406 may be programmed to analyze the signal from the monitoring component 402 to determine whether the brain is exhibiting a symptom of a neurological disorder. The processor 406 may transmit the instruction to the stimulation component 404 to apply to the brain the acoustic signal in response to determining that the brain is exhibiting the symptom of the neurological disorder. The acoustic signal may suppress the symptom of the neurological disorder. For example, the symptom may be a seizure, and the neurological disorder may be one or more of stroke, Parkinson's disease, migraine, tremors, frontotemporal dementia, traumatic brain injury, depression, anxiety, Alzheimer's disease, dementia, multiple sclerosis, schizophrenia, brain damage, neurodegeneration, central nervous system (CNS) disease, encephalopathy, Huntington's disease, autism, attention deficit hyperactivity disorder (ADHD), amyotrophic lateral sclerosis (ALS), and concussion.

In some embodiments, the software to program the ultrasound transducers may send real-time sensor readings (e.g., from EEG sensors, accelerometers, EKG sensors, and/or other suitable sensors) to a processor running machine learning algorithms continuously, e.g., a deep learning network as described with respect to FIGS. 11B and 11C. For example, this processor may be local, on the device itself, or in the cloud. These machine learning algorithms executing on the processor may perform three tasks: 1) detect when a seizure is present, 2) predict when a seizure is likely to occur within the near future (e.g., within one hour), and 3) output a location to aim the stimulating ultrasound beam. Immediately after the processor detects that a seizure has begun, the stimulating ultrasound beam may be turned on and aimed at the location determined by the output of the algorithm(s). For patients with seizures that always have the same characteristics/focus, it is likely that once a good beam location is found, it may not change. Another example for how the beam may be activated is when the processor predicts that a seizure is likely to occur in the near future, the beam may be turned on at a relatively low intensity (e.g., relative to the intensity used when a seizure is detected). In some embodiments, the target for the stimulating ultrasound beam may not be the seizure focus itself. For example, the target may be a seizure “choke point,” i.e., a location outside of the seizure focus that when stimulated can shut down seizure activity.

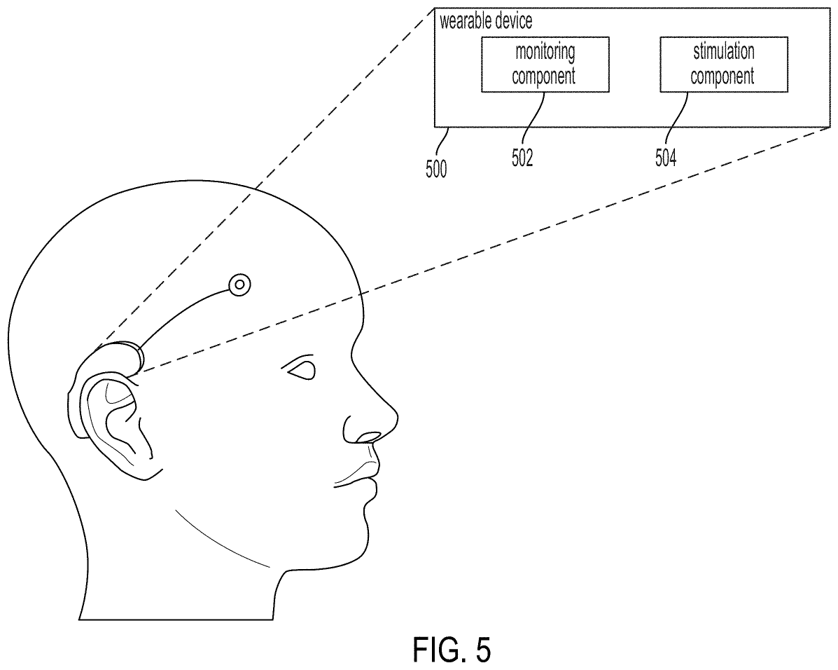

FIG. 5 shows a block diagram for a wearable device 500 for substantially non-destructive acoustic stimulation, in accordance with some embodiments of the technology described herein. The device 500 is wearable by a person and includes a monitoring component 502 and a stimulation component 504. The monitoring component 502 and/or the stimulation component 504 may be disposed on the head of the person in a non-invasive manner.

The monitoring component 502 may include a sensor that is configured to detect a signal, e.g., an electrical signal, a mechanical signal, an optical signal, an infrared signal, or another suitable type of signal, from the brain of the person. For example, the sensor may be an electroencephalogram (EEG) sensor, and the signal may be an EEG signal. The stimulation component 504 may include an ultrasound transducer configured to apply to the brain an ultrasound signal that has a low power density, e.g., between 1 and 100 watts/cm², and is substantially non-destructive with respect to tissue when applied to the brain. For example, the ultrasound signal may have a frequency between 100 kHz and 1 MHz, a spatial resolution between 0.001 cm' and 0.1 cm³, and/or the low power density between 1 and 100 watts/cm² as measured by spatial-peak pulse-average intensity. The ultrasound signal may suppress the symptom of the neurological disorder. For example, the symptom may be a seizure, and the neurological disorder may be epilepsy or another suitable neurological disorder.

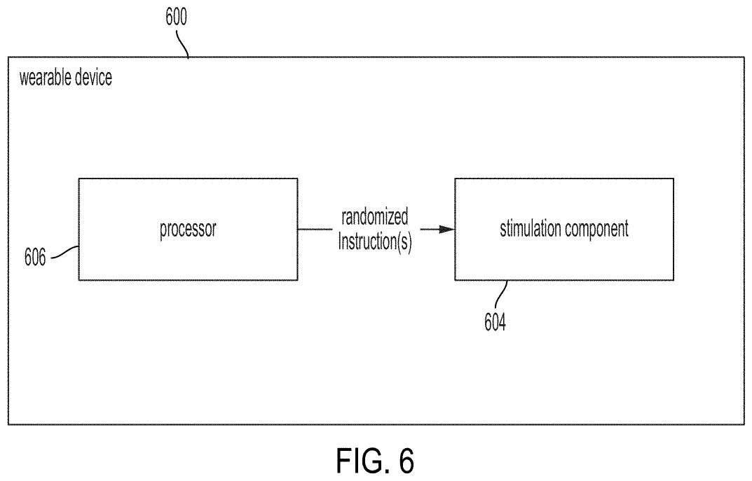

FIG. 6 shows a block diagram for a wearable device 600 for acoustic stimulation, e.g., randomized acoustic stimulation, in accordance with some embodiments of the technology described herein. The device 600 is wearable by a person and includes a stimulation component 604 and a processor 606. The stimulation component 604 may include a transducer that is configured to apply to the brain of the person acoustic signals. For example, the transducer may be an ultrasound transducer, and the acoustic signal may be an ultrasound signal. In some embodiments, the ultrasound signal may have a low power density and be substantially non-destructive with respect to tissue when applied to the brain. In some embodiments, the transducer may be disposed on the head of the person in a non-invasive manner.

In some embodiments, the processor 606 may transmit an instruction to the stimulation component 604 to activate the brain tissue at random intervals, e.g., sporadically throughout the day and/or night, thereby preventing the brain from settling into a seizure state. For example, for patients with generalized epilepsy, the device 600 may stimulate the thalamus or another suitable region of the brain at random times throughout the day and/or night, e.g., around every 10 minutes. In some embodiments, the stimulation component 604 may include another transducer. The device 600 and/or the processor 606 may select one of the transducers to apply to the brain the acoustic signal at one or more random intervals.

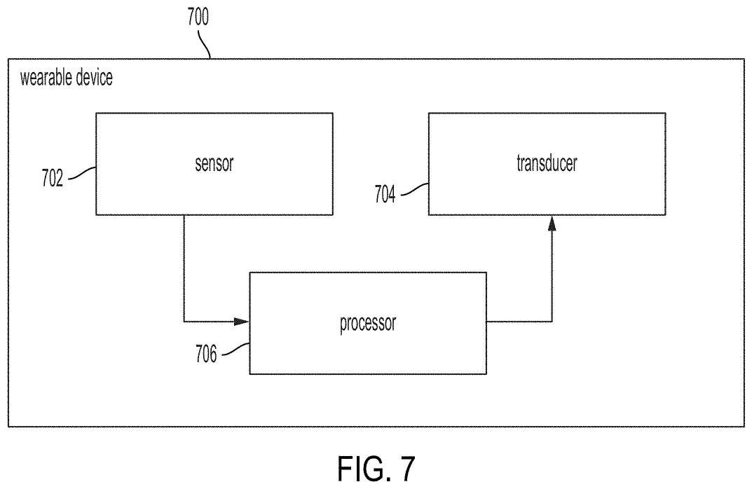

FIG. 7 shows a block diagram for a wearable device 700 for treating a neurological disorder using ultrasound stimulation, in accordance with some embodiments of the technology described herein. The device 700 is wearable by (or attached to or implanted within) a person and can be used to treat epileptic seizures. The device 700 includes a sensor 702, a transducer 704, and a processor 706. The sensor 702 may be configured to detect an EEG signal from the brain of the person. The transducer 704 may be configured to apply to the brain a low power, substantially non-destructive ultrasound signal. The ultrasound signal may suppress one or more epileptic seizures. For example, the ultrasound signal may have a frequency between 100 kHz and 1 MHz, a spatial resolution between 0.001 cm³ and 0.1 cm³, and/or a power density between 1 and 100 watts/cm² as measured by spatial-peak pulse-average intensity. In some embodiments, the sensor and the transducer may be disposed on the head of the person in a non-invasive manner.

The processor 706 may be in communication with the sensor 702 and the transducer 704. The processor 706 may be programmed to receive, from the sensor 702, the EEG signal detected from the brain and transmit an instruction to the transducer 704 to apply to the brain the ultrasound signal. In some embodiments, the processor 706 may be programmed to analyze the EEG signal to determine whether the brain is exhibiting an epileptic seizure and, in response to determining that the brain is exhibiting the epileptic seizure, transmit the instruction to the transducer 704 to apply to the brain the ultrasound signal.

In some embodiments, the processor 706 may be programmed to transmit an instruction to the transducer 704 to apply to the brain the ultrasound signal at one or more random intervals. In some embodiments, the transducer 704 may include two or more transducers, and the processor 706 may be programmed to select one of the transducers to transmit an instruction to apply to the brain the ultrasound signal at one or more random intervals.

Closed-Loop System using Machine Learning to Steer Focus of Ultrasound Beam within Human Brain

Conventional brain-machine interfaces are limited in that the brain regions that receive stimulation may not be changed in real time. This may be problematic because it is often difficult to locate an appropriate brain region to stimulate in order to treat symptoms of neurological disorders. For example, in epilepsy, it may not be clear which region within the brain should be stimulated to suppress or stop a seizure. The appropriate brain region may be the seizure focus (which can be difficult to localize), a region that may serve to suppress the seizure, or another suitable brain region. Conventional solutions, such as implantable electronic responsive neural stimulators and deep brain stimulators, can only be positioned once by doctors taking their best guess or choosing some pre-determined region of the brain. Therefore, brain regions that can receive stimulation cannot be changed in real time in conventional systems.

The inventors have appreciated that treatment for neurological disorders may be more effective when the brain region of the stimulation may be changed in real time, and in particular, when the brain region may be changed remotely. Because the brain region may be changed in real time and/or remotely, tens (or more) of locations per second may be tried, thereby closing in on the appropriate brain region for stimulation quickly with respect to the duration of an average seizure. Such a treatment may be achievable using ultrasound to stimulate the brain. In some embodiments, the patient may wear an array of ultrasound transducers (e.g., such an array is placed on the scalp of the person), and an ultrasound beam may be steered using beamforming methods such as phased arrays. In some embodiments, with wedge transducers, fewer number of transducers may be used. In some embodiments, with wedge transducers, the device may be more energy efficient due to lower power requirements of the wedge transducers. U.S. Patent Application Publication No. 2018/0280735 provides further information on exemplary embodiments of the wedge transducers, the entirety of which incorporated by reference herein. The target of the beam may be changed by programming the array. If stimulation in a certain brain region is not working, the beam may be moved to another region of the brain to try again, at no harm to the patient.

In some embodiments, a machine learning algorithm that senses the brain state may be connected to the beam steering algorithm to make a closed-loop system, e.g., including a deep learning network. The machine learning algorithm that senses the brain state may take as input recordings from EEG sensors, EKG sensors, accelerometers, and/or other suitable sensors. Various filters may be applied to these combined inputs, and the outputs of these filters may be combined in a generally nonlinear fashion, to extract a useful representation of the data. Then, a classifier may be trained on this high-level representation. This may be accomplished using deep learning and/or by pre-specifying the filters and training a classifier, such as a Support Vector Machine (SVM). In some embodiments, the machine learning algorithm may include training a recurrent neural network (RNN), such as a long short-term memory (LSTM) unit based RNN, to map the high-dimensional input data into a smoothly-varying trajectory through a latent space representative of a higher-level brain state. These machine learning algorithms executing on the processor may perform three tasks: 1) detect when a symptom of a neurological disorder is present, e.g., a seizure, 2) predict when a symptom is likely to occur within the near future (e.g., within one hour), and 3) output a location to aim the stimulating acoustic signal, e.g., an ultrasound beam. Any or all of these tasks may be performed using a deep learning network or another suitable network. More details regarding this technique are described later in this disclosure, including with respect to FIGS. 11B and 11C.

Taking the example of epilepsy, the goal may be to suppress or stop a seizure that has already started. In this example, the closed-loop system may work as follows. First, the system may execute a measurement algorithm that measures the “strength” of seizure activity, with the beam positioned in some preset initial location (for example, the hippocampus for patients with temporal lobe epilepsy). The beam location may then be slightly changed and the resulting change in seizure strength may be measured using the measurement algorithm. If the seizure activity has reduced, the system may continue moving the beam in this direction. If the seizure activity has increased, the system may move the beam in the opposite or a different direction. Because the beam location may be programmed electronically, tens of beam locations per second may be tried, thereby closing in on the appropriate stimulation location quickly with respect to the duration of an average seizure.

In some embodiments, some brain regions may be inappropriate for stimulation. For example, stimulating parts of the brain stem may lead to irreversible damage or discomfort. In this case, the closed-loop system may follow a “constrained” gradient descent solution where the appropriate stimulation location is taken from a set of feasible points. This may ensure that the off-limit brain regions are never stimulated.

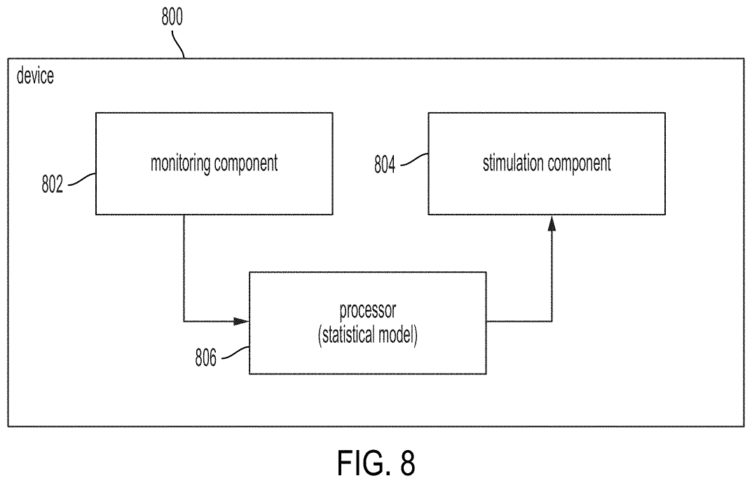

FIG. 8 shows a block diagram for a device 800 to steer acoustic stimulation, in accordance with some embodiments of the technology described herein. The device 800, e.g., a wearable device, may be part of a closed-loop system that uses machine learning to steer focus of an ultrasound beam within the brain. The device 800 may include a monitoring component 802, e.g., a sensor, that is configured to detect a signal, e.g., an electrical signal, a mechanical signal, an optical signal, an infrared signal, or another suitable type of signal, from the brain of the person. For example, the sensor may be an EEG sensor, and the signal may be an electrical signal, such as an EEG signal. The device 800 may include a stimulation component 804, e.g., a set of transducers, each configured to apply to the brain an acoustic signal. For example, one or more of the transducers may be an ultrasound transducer, and the acoustic signal may be an ultrasound signal. The sensor and/or the set of transducers may be disposed on the head of the person in a non-invasive manner. In some embodiments, the device 800 may include a processor 806 in communication with the sensor and the set of transducers. The processor 806 may select one of the transducers using a statistical model trained on data from prior signals detected from the brain. For example, data from prior signals detected from the brain may be accessed from an electronic health record of the person.

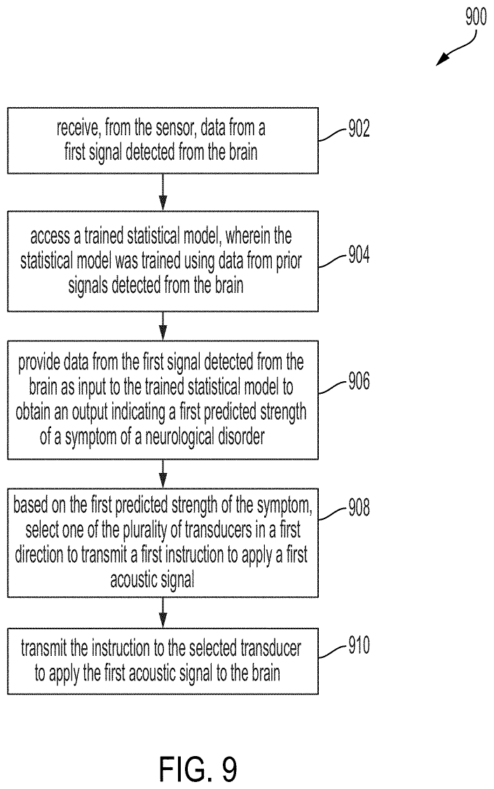

FIG. 9 shows a flow diagram 900 for a device to steer acoustic stimulation, in accordance with some embodiments of the technology described herein.

At 902, the processor, e.g., processor 806, may receive, from the sensor, data from a first signal detected from the brain.

At 904, the processor may access a trained statistical model. The statistical model may be trained using data from prior signals detected from the brain. For example, the statistical model may include a deep learning network trained using data from the prior signals detected from the brain.

At 906, the processor may provide data from the first signal detected from the brain as input to the trained statistical model, e.g., a deep learning network, to obtain an output indicating a first predicted strength of a symptom of a neurological disorder, e.g., an epileptic seizure.

At 908, based on the first predicted strength of the symptom, the processor may select one of the transducers in a first direction to transmit a first instruction to apply a first acoustic signal. For example, the first acoustic signal may be an ultrasound signal that has a low power density, e.g., between 1 and 100 watts/cm², and is substantially non-destructive with respect to tissue when applied to the brain. The acoustic signal may suppress the symptom of the neurological disorder.

At 910, the processor may transmit the instruction to the selected transducer to apply the first acoustic signal to the brain.

In some embodiments, the processor may be programmed to provide data from a second signal detected from the brain as input to the trained statistical model to obtain an output indicating a second predicted strength of the symptom of the neurological disorder. If it is determined that the second predicted strength is less than the first predicted strength, the processor may select one of the transducers in the first direction to transmit a second instruction to apply a second acoustic signal. If it is determined that the second predicted strength is greater than the first predicted strength, the processor may select one of the transducers in a direction opposite to or different from the first direction to transmit the second instruction to apply the second acoustic signal.

### Novel Detection Algorithms

Conventional approaches consider seizure detection to be a classification problem. For example, a window of EEG data (e.g., 5 seconds long) may be fed into a classifier which outputs a binary label representing whether or not the input is from a seizure. Running the algorithm in real time may entail running the algorithm on consecutive windows of EEG data. However, the inventors have discovered that there is nothing in such an algorithm structure, or in the training of the algorithm, to accommodate that the brain does not quickly switch back and forth between seizure and non-seizure. If the current window is a seizure, there is a high probability that the next window will be a seizure too. This reasoning will only fail for the very end of the seizure. Similarly, if the current window is not a seizure, there is a high probability that the next window will also not be a seizure. This reasoning will only fail for the very beginning of the seizure. The inventors have appreciated that it would be preferable to reflect the “smoothness” of seizure state in the structure of the algorithm or in the training by penalizing network outputs that oscillate on short time scales. The inventors have accomplished this by, for example, adding a regularization term to the loss function that is proportional to the total variation of the outputs, or the L1/L2 norm of the derivative (computed via finite difference) of the outputs, or the L1/L2 norm of the second derivative of the outputs. In some embodiments, RNNs with LSTM units may automatically give smooth output. In some embodiments, a way to achieve smoothness of the detection outputs may be to train a conventional, non-smooth detection algorithm, and feed its results into a causal low-pass filter, and using this low-pass filtered output as the final result. This may ensure that the final result is smooth. For example, the non-smooth detection algorithm may use one or both of the following equations to generate the final result:

$$\begin{matrix}L ( w ) = {\sum }_{i = 1}^{n} {‖ y [ i ] - {\hat{y}}_{w} [ i ] ‖}^{2} + λ {‖ {\hat{y}}_{w} [ i ] ‖}_{TV} & ( 1 ) \\ L ( w ) = {\sum }_{i = 1}^{n} {‖ y [ i ] - {\hat{y}}_{w} [ i ] ‖}^{2} + λ \vert {\hat{y}}_{w} [ i ] - {\hat{y}}_{w} [ i - 1 ] \vert & ( 2 )\end{matrix}$$

In equations (1) and (2), y[i] is the ground-truth label of seizure, or no seizure, for sample i, ŷ_{w}[i] is the output of the algorithm for sample i. L(w) is the machine learning loss function evaluated at the model parameterized by w (meant to represent the weights in a network). The first term in L(w) may measure how accurately the algorithm classifies seizures. The second term in L(w) (multiplied by λ) is a regularization term that may encourage the algorithm to learn solutions that change smoothly over time. Equations (1) and (2) are two examples for regularization as shown. Equation (1) is the total variation (TV) norm, and equation (2) is the absolute value of the first derivative. Both equations may try to enforce smoothness. In equation (1), the TV norm may be small for a smooth output and large for an output that is not smooth. In equation (2), the absolute value of the first derivative is penalized to try to enforce smoothness. In certain cases, equation (1) may work better than equation (2), or vice versa, the results of which may be determined empirically by training a conventional, non-smooth detection algorithm using equation (1) and comparing the final result to a similar algorithm trained using equation (2).

Conventionally, EEG data is annotated in a binary fashion, so that one moment is classified as not a seizure and the next is classified as a seizure. The exact seizure start and end times are relatively arbitrary because there may not be an objective way to locate the beginning and end of a seizure. However, using conventional algorithms, the detection algorithm may be penalized for not perfectly agreeing with the annotation. The inventors have appreciated that it may be better to “smoothly” annotate the data, e.g., using smooth window labels that rise from 0 to 1 and fall smoothly from 1 back to 0, with 0 representing a non-seizure and 1 representing a seizure. This annotation scheme may better reflect that seizures evolve over time and that there may be ambiguity involved in the precise demarcation. Accordingly, the inventors have applied this annotation scheme to recast seizure detection from a detection problem to a regression machine learning problem.

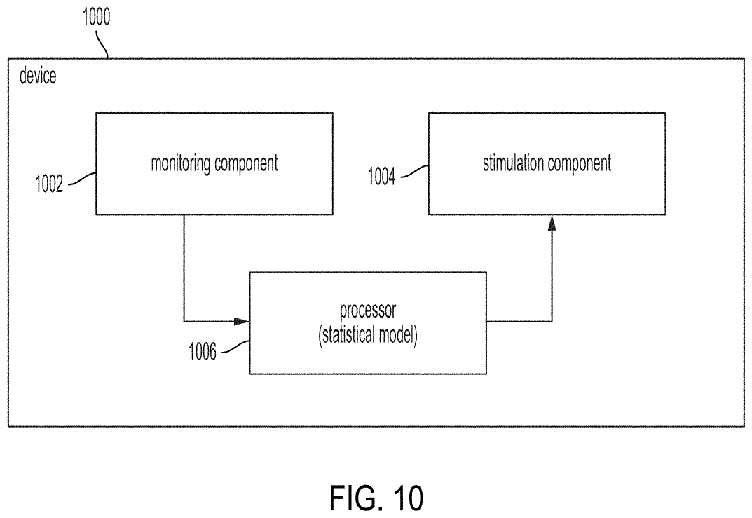

FIG. 10 shows a block diagram for a device using a statistical model trained on annotated signal data, in accordance with some embodiments of the technology described herein. The statistical model may include a deep learning network or another suitable model. The device 1000, e.g., a wearable device, may include a monitoring component 1002, e.g., a sensor, that is configured to detect a signal, e.g., an electrical signal, a mechanical signal, an optical signal, an infrared signal, or another suitable type of signal, from the brain of the person. For example, the sensor may be an EEG sensor, and the signal may be an EEG signal. The device 1000 may include a stimulation component 1004, e.g., a set of transducers, each configured to apply to the brain an acoustic signal. For example, one or more of the transducers may be an ultrasound transducer, and the acoustic signal may be an ultrasound signal. The sensor and/or the set of transducers may be disposed on the head of the person in a non-invasive manner.

In some embodiments, the device 1000 may include a processor 1006 in communication with the sensor and the set of transducers. The processor 1006 may select one of the transducers using a statistical model trained on signal data annotated with one or more values relating to identifying a health condition. e.g., respective values relating to increasing strength of a symptom of a neurological disorder. For example, the signal data may include data from prior signals detected from the brain and may be accessed from an electronic health record of the person. In some embodiments, the statistical model may be trained on data from prior signals detected from the brain annotated with the respective values, e.g., between 0 and 1, relating to increasing strength of the symptom of the neurological disorder. In some embodiments, the statistical model may include a loss function having a regularization term that is proportional to a variation of outputs of the statistical model, an L1/L2 norm of a derivative of the outputs, or an L1/L2 norm of a second derivative of the outputs.

FIG. 11A shows a flow diagram 1100 for a device using a statistical model trained on annotated signal data, in accordance with some embodiments of the technology described herein.

At 1102, the processor, e.g., processor 1006, may receive, from the sensor, data from a first signal detected from the brain.

At 1104, the processor may access a trained statistical model, wherein the statistical model was trained using data from prior signals detected from the brain annotated with one or more values relating to identifying a health condition, e.g., respective values (e.g., between 0 and 1) relating to increasing strength of a symptom of a neurological disorder.

At 1106, the processor may provide data from the first signal detected from the brain as input to the trained statistical model to obtain an output indicating a first predicted strength of the symptom of the neurological disorder, e.g., an epileptic seizure.

At 1108, based on the first predicted strength of the symptom, the processor may select one of the plurality of transducers in a first direction to transmit a first instruction to apply a first acoustic signal.

At 1110, the processor may transmit the instruction to the selected transducer to apply the first acoustic signal to the brain. For example, the first acoustic signal may be an ultrasound signal that has a low power density, e.g., between 1 and 100 watts/cm², and is substantially non-destructive with respect to tissue when applied to the brain. The acoustic signal may suppress the symptom of the neurological disorder.

In some embodiments, the processor may be programmed to provide data from a second signal detected from the brain as input to the trained statistical model to obtain an output indicating a second predicted strength of the symptom of the neurological disorder. If it is determined that the second predicted strength is less than the first predicted strength, the processor may select one of the transducers in the first direction to transmit a second instruction to apply a second acoustic signal. If it is determined that the second predicted strength is greater than the first predicted strength, the processor may select one of the transducers in a direction opposite to or different from the first direction to transmit the second instruction to apply the second acoustic signal.

In some embodiments, the inventors have developed a deep learning network to detect one or more other symptoms of a neurological disorder. For example, the deep learning network may be used to predict seizures. The deep learning network includes a Deep Convolutional Neural Network (DCNN), which embeds or encodes the data onto a n-dimensional representation space (e.g., 16-dimensional) and a Recurrent Neural Network (RNN), which computes detection scores by observing changes in the representation space through time. However, the deep learning network is not so limited and may include alternative or additional architectural components suitable for predicting one or more symptoms of a neurological disorder.

In some embodiments, the features that are provided as input to the deep learning network may be received and/or transformed in the time domain or the frequency domain. Some embodiments, a network trained using frequency domain-based features may output more accurate predictions compared to another network trained using time domain-based features. For example, a network trained using frequency domain-based features may output more accurate predictions because the wave shape induced in EEG signal data captured during a seizure may have temporally limited exposure. Accordingly, a discrete wavelet transform (DWT), e.g., with the Daubechies 4-tab (db-4) mother wavelet or another suitable wavelet, may be used to transform the EEG signal data into the frequency domain. Other suitable wavelet transforms may be used additionally or alternatively in order to transform the EEG signal data into a form suitable for input to the deep learning network. In some embodiments, one-second windows of EEG signal data at each channel may be chosen and the DWT may be applied up to 5 levels, or another suitable number of levels. In this case, each batch input to the deep learning network may be a tensor with dimensions equal to (batch size x sampling frequency x number of EEG channels x DWT levels+1). This tensor may be provided to the DCNN encoder of the deep learning network.

In some embodiments, signal statistics may he different for different people and may change over time even for a particular person. Hence, the network may be highly susceptible to overfitting especially when the provided training data is not large enough. This information may be utilized in developing the training framework for the network such that the DCNN encoder can embed the signal onto a space in which at least temporal drifts convey information about seizure. During the training, one or more objective functions may be used to fit the DCNN encoder, including a Siamese loss and a classification loss, which are further described below.

1. Siamese loss: In one-shot or few-shot learning frameworks, i.e., frameworks with small training data sets, a Siamese loss based network may be designed to indicate a pair of input instances are from the same category or not. The setup in the network may be aimed to detect if two temporally close samples are both from the same category or not in the same patient.

Classification loss: Binary-cross entropy is a widely used objective function for supervised learning. This objective function may be used to decrease the distance among embeddings from the same category while increasing the distance between classes as much as possible, regardless of piece-wise behavior and subjectivity of EEG signal statistics. The paired data segments mat help to increase sample comparisons quadratically and hence mitigate the overfitting caused by lack of data.

In some embodiments, each time a batch of training data is formed, the onset of one-second windows may be selected randomly to help with data augmentation, thereby increasing the size of the training data.

In some embodiments, the DCNN encoder may include a 13-layer 2-D convolutional neural network with fractional max-pooling (FMP). After training the DCNN encoder, the weights of this network may be fixed. The output from the DCNN encoder may then be used as an input layer to an RNN for final detection. In some embodiments, the RNN may include a bidirectional-LSTM followed by two fully connected neural network layers. In one example, the RNN may be trained by feeding 30 one-second frequency domain EEG signal samples to the DCNN encoder and then the resulting output to the RNN at each trial.

In some embodiments, data augmentation and/or statistical inference may help to reduce estimation error for the deep learning network. In one example, for the setup proposed for this deep learning network, each 30-second time window may be evaluated multiple times by adding jitter to the onset of one-second time windows. The number of sampling may depend on computational capacity. For example, for the described setup, real time capability may be maintained with up to 30 times of Monte-Carlo simulations.

It should be appreciated that the described deep learning network is only one example implementation and that other implementations may be employed. For example, in some embodiments, one or more other types of neural network layers may be included in the deep learning network instead of or in addition to one or more of the layers in the described architecture. For example, in some embodiments, one or more convolutional, transpose convolutional, pooling, unpooling layers, and/or batch normalization may be included in the deep learning network. As another example, the architecture may include one or more layers to perform a nonlinear transformation between pairs of adjacent layers. The non-linear transformation may be a rectified linear unit (ReLU) transformation, a sigmoid, and/or any other suitable type of non-linear transformation, as aspects of the technology described herein are not limited in this respect.

As another example of a variation, in some embodiments, any other suitable type of recurrent neural network architecture may be used instead of or in addition to an LSTM architecture.

It should also be appreciated that although in the described architecture illustrative dimensions are provided for the inputs and outputs for the various layers, these dimensions are for illustrative purposes only and other dimensions may be used in other embodiments.

Any suitable optimization technique may be used for estimating neural network parameters from training data. For example, one or more of the following optimization techniques may be used: stochastic gradient descent (SGD), mini-batch gradient descent, momentum SGD, Nesterov accelerated gradient, Adagrad, Adadelta, RMSprop, Adaptive Moment Estimation (Adam), AdaMax, Nesterov-accelerated Adaptive Moment Estimation (Nadam), AMSGrad.

FIG. 11B shows a convolutional neural network 1150 that may be used to detect one or more symptoms of a neurological disorder, in accordance with some embodiments of the technology described herein. The deep learning network described herein may include the convolutional neural network 1150, and additionally or alternatively another type of network, suitable for detecting whether the brain is exhibiting a symptom of a neurological disorder and/or for guiding transmission of an acoustic signal to a region of the brain. For example, convolutional neural network 1150 may be used to detect a seizure and/or predict a location of the brain to transmit an ultrasound signal. As shown, the convolutional neural network comprises an input layer 1154 configured to receive information about the input 1152 (e.g., a tensor), an output layer 1158 configured to provide the output (e.g., classifications in an n-dimensional representation space), and a plurality of hidden layers 1156 connected between the input layer 1154 and the output layer 1158. The plurality of hidden layers 1156 include convolution and pooling layers 1160 and fully connected layers 1162.

The input layer 1154 may be followed by one or more convolution and pooling layers 1160. A convolutional layer may comprise a set of filters that are spatially smaller (e.g., have a smaller width and/or height) than the input to the convolutional layer (e.g., the input 1152). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 1160 may be followed by fully connected layers 1162. The fully connected layers 1162 may comprise one or more layers each with one or more neurons that receives an input from a previous layer (e.g., a convolutional or pooling layer) and provides an output to a subsequent layer (e.g., the output layer 1158). The fully connected layers 1162 may be described as “dense” because each of the neurons in a given layer may receive an input from each neuron in a previous layer and provide an output to each neuron in a subsequent layer. The fully connected layers 1162 may be followed by an output layer 1158 that provides the output of the convolutional neural network. The output may be, for example, an indication of which class, from a set of classes, the input 1152 (or any portion of the input 1152) belongs to. The convolutional neural network may be trained using a stochastic gradient descent type algorithm or another suitable algorithm. The convolutional neural network may continue to be trained until the accuracy on a validation set (e.g., a held out portion from the training data) saturates or using any other suitable criterion or criteria.

It should be appreciated that the convolutional neural network shown in FIG. 11B is only one example implementation and that other implementations may be employed. For example, one or more layers may be added to or removed from the convolutional neural network shown in FIG. 11B. Additional example layers that may be added to the convolutional neural network include: a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output.

Convolutional neural networks may be employed to perform any of a variety of functions described herein. It should be appreciated that more than one convolutional neural network may be employed to make predictions in some embodiments. The first and second neural networks may comprise a different arrangement of layers and/or be trained using different training data.

FIG. 11C shows an exemplary interface 1170 including predictions from a deep learning network, in accordance with some embodiments of the technology described herein. The interface 1170 may be generated for display on a computing device, e.g., computing device 308 or another suitable device. A wearable device, a mobile device, and/or another suitable device may provide one or more signals detected from the brain, e.g., an EEG signal or another suitable signal, to the computing device. For example, the interface 1170 shows signal data 1172 including EEG signal data. This signal data may be used to train a deep learning network to determine whether the brain is exhibiting a symptom of a neurological disorder, e.g., a seizure or another suitable symptom. The interface 1170 further shows EEG signal data 1174 with predicted seizures and doctor annotations indicating a seizure. The predicted seizures may be determined based on an output from the deep learning network. The inventors have developed such deep learning networks for detecting seizures and have found the predictions to closely correspond to annotations from a neurologist. For example, as indicated in FIG. 11C, the spikes 1178, which indicate predicted seizures, are found to be overlapping or nearly overlapping with doctor annotations 1176 indicating a seizure.

The computing device, the mobile device, or another suitable device may generate a portion of the interface 1170 to warn the person and/or a caretaker when the person is likely to have a seizure and/or when the person will be seizure-free. The interface 1170 generated on a mobile device, e.g., mobile device 304, and/or a computing device, computing device 308, may display an indication 1180 or 1182 for whether a seizure is detected or not. For example, the mobile device may display real-time seizure risk for a person suffering from a neurological disorder. In the event of a seizure, the mobile device may alert the person, a caregiver, or another suitable entity. For example, the mobile device may inform a caretaker that a seizure is predicted in the next 30 minutes, next hour, or another suitable time period. In another example, the mobile device may send alerts to the caretaker when a seizure does occur and/or record seizure activity, such as signals from the brain, for the caretaker to refine treatment of the person's neurological disorder.

Tiered algorithms to optimize power consumption and performance

The inventors have appreciated that, to enable a device to be functional with long durations in between battery charges, it may be necessary to reduce power consumption as much as possible. There may be at least two activities that dominate power consumption:
1. Running machine learning algorithms, e.g., a deep learning network, to classify brain state based on physiological measurements (e.g., seizure vs. not seizure, or measure risk of having seizure in near future, etc.); and/or
2. Transmitting data from the device to a mobile phone or to a server for further processing and/or executing machine learning algorithms on the data.

In some embodiments, less computationally intensive algorithms may be run on the device, e.g., a wearable device, and when the output of the algorithms) exceeds a specified threshold, the device may, e.g., turn on the radio, and transmit the relevant data to a mobile phone or a server, e.g., a cloud server, for further processing via more computationally intensive algorithms. Taking the example of seizure detection, a more computationally intensive or heavyweight algorithm may have a low false-positive rate and a low false-negative rate. To obtain a less computationally intensive or lightweight algorithm, one rate or the other may be sacrificed. The inventors have appreciated that the key is to allow for more false positives, i.e., a detection algorithm with high sensitivity (e.g., never misses a true seizure) and low specificity e.g., many false-positives, often labels data as a seizure when there is no seizure). Whenever the device's lightweight algorithm labels data as a seizure, the device may transmit the data to the mobile device or the cloud server to execute the heavyweight algorithm. The device may receive the results of the heavyweight algorithm, and display these results to the user. In this way, the lightweight algorithm on the device may act as a filter that drastically reduces the amount of power consumed, e.g., by reducing computation power and/or the amount of data transmitted, while maintaining the predictive performance of the whole system including the device, the mobile phone, and/or the cloud server.

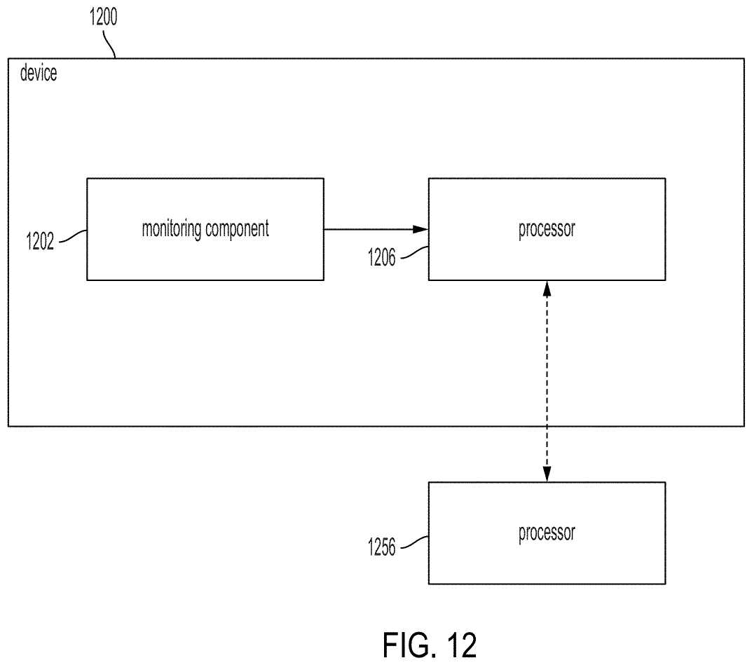

FIG. 12 shows a block diagram for a device for energy efficient monitoring of the brain, in accordance with some embodiments of the technology described herein. The device 1200, e.g., a wearable device, may include a monitoring component 1202, e.g., a sensor, that is configured to detect an signal, e.g., an electrical signal, a mechanical signal, an optical signal, an infrared signal, or another suitable type of signal, from the brain of the person. For example, the sensor may be an EEG sensor, and the signal may be an electrical signal, such as an EEG signal. The sensor may be disposed on the head of the person in a non-invasive manner.

The device 1200 may include a processor 1206 in communication with the sensor. The processor 1206 may be programmed to identify a health condition, e.g., predict a strength of a symptom of a neurological disorder, and, based on the identified health condition, e.g., predicted strength, provide data from the signal to a processor 1256 outside the device 1200 to corroborate or contradict the identified health condition, e.g., predicted strength.

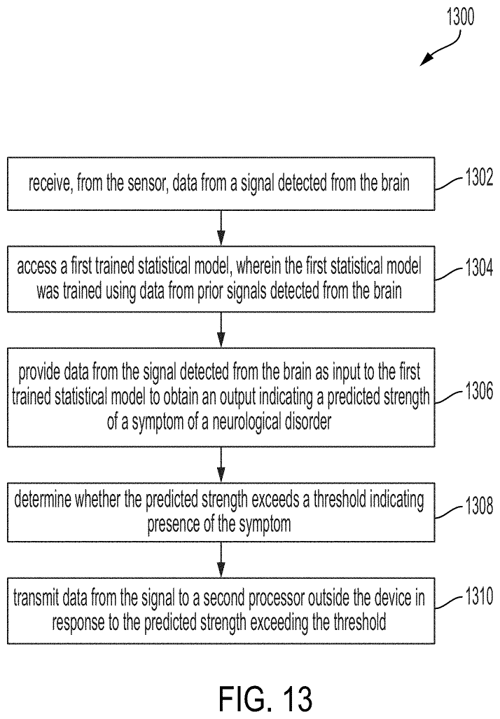

FIG. 13 shows a flow diagram 1300 for a device for energy efficient monitoring of the brain, in accordance with some embodiments of the technology described herein.

At 1302, the processor, e.g., processor 1206, may receive, from the sensor, data from the signal detected from the brain.

At 1304, the processor may access a first trained statistical model. The first statistical model may be trained using data from prior signals detected from the brain.

At 1306, the processor may provide data from the signal detected from the brain as input to the first trained statistical model to obtain an output identifying a health condition, e.g., indicating a predicted strength of a symptom of a neurological disorder.

At 1308, the processor may determine whether the predicted strength exceeds a threshold indicating presence of the symptom.

At 1310, in response to the predicted strength exceeding the threshold, the processor may transmit data from the signal to a second processor outside the device. In some embodiments, the second processor, e.g., processor 1256, may be programmed to provide data from the signal to a second trained statistical model to obtain an output to corroborate or contradict the identified health condition, e.g., the predicted strength of the symptom.

In some embodiments, the first trained statistical model be trained to have high sensitivity and low specificity. In some embodiments, the second trained statistical model may be trained to have high sensitivity and high specificity. Therefore the first processor using the first trained statistical model may use a smaller amount of power than the first processor using the second trained statistical model.

### Example Computer Architecture

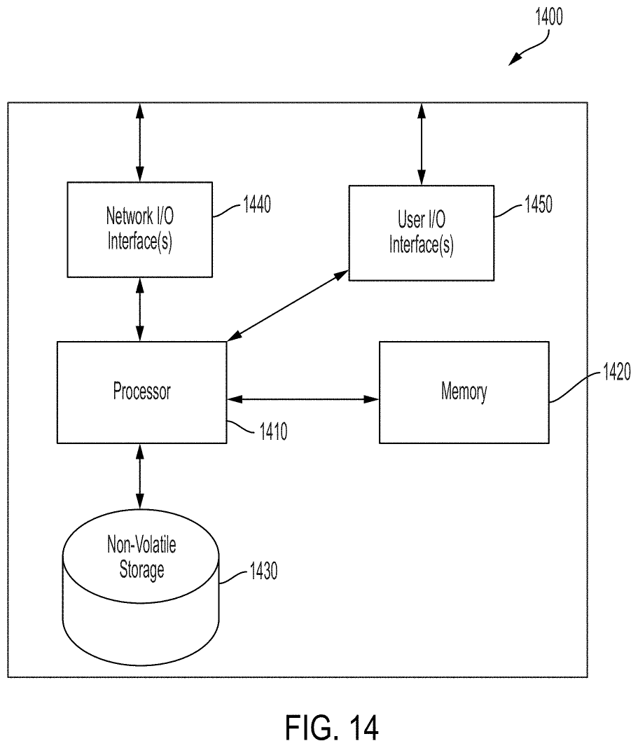

An illustrative implementation of a computer system 1400 that may be used in connection with any of the embodiments of the technology described herein is shown in FIG. 14. The computer system 1400 includes one or more processors 1410 and one or more articles of manufacture that comprise non-transitory computer-readable storage media (e.g., memory 1420 and one or more non-volatile storage media 1430). The processor 1410 may control writing data to and reading data from the memory 1420 and the non-volatile storage device 1430 in any suitable manner, as the aspects of the technology described herein are not limited in this respect. To perform any of the functionality described herein, the processor 1410 may execute one or more processor-executable instructions stored in one or more non-transitory computer-readable storage media (e.g., the memory 1420), which may serve as non-transitory computer-readable storage media storing processor-executable instructions for execution by the processor 1410.

Computing device 1400 may also include a network input/output (I/O) interface 1440 via which the computing device may communicate with other computing devices (e.g., over a network), and may also include one or more user I/O interfaces 1450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or”should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

Some aspects of the technology described herein may be understood further based on the non-limiting illustrative embodiments described below in the Appendix. While some aspects in the Appendix, as well as other embodiments described herein, are described with respect to treating seizures for epilepsy, these aspects and/or embodiments may be equally applicable to treating symptoms for any suitable neurological disorder. Any limitations of the embodiments described below in the Appendix are limitations only of the embodiments described in the Appendix, and are not limitations of any other embodiments described herein.

What is claimed is:

1. A device wearable by a person, comprising:
a transducer configured to apply to the brain of the person acoustic signals.

2. The device as claimed in claim 1, wherein the transducer is configured to apply to the brain of the person acoustic signals randomly.

3. The device as claimed in claim 1, wherein the transducer includes an ultrasound transducer, and wherein the acoustic signals include an ultrasound signal.

4. The device as claimed in claim 3, wherein the ultrasound signal has a frequency between 100 kHz and 1 MHz, a spatial resolution between 0.001 cm³ and 0.1 cm³, and/or a power density between 1 and 100 watts/cm² as measured by spatial-peak pulse-average intensity.

5. The device as claimed in claim 3, wherein the ultrasound signal has a low power density and is substantially non-destructive with respect to tissue when applied to the brain.

6. The device as claimed in claim 1, wherein the transducer is disposed on the head of the person in a non-invasive manner.

7. The device as claimed in claim 1, wherein the acoustic signal suppresses a symptom of a neurological disorder.

8. The device as claimed in claim 7, wherein the neurological disorder includes one or more of stroke, Parkinson's disease, migraine, tremors, frontotemporal dementia, traumatic brain injury, depression, anxiety, Alzheimer's disease, dementia, multiple sclerosis, schizophrenia, brain damage, neurodegeneration, central nervous system (CNS) disease, encephalopathy, Huntington's disease, autism, attention deficit hyperactivity disorder (AMID), amyotrophic lateral sclerosis (ALS), and concussion.

9. The device as claimed in claim 7, wherein the symptom includes a seizure.

10. A method for operating a device wearable by a person, the device including a transducer, comprising:
applying to the brain of the person acoustic signals.

11. An apparatus comprising:
a device worn by or attached to a person including a transducer configured to apply to the brain of the person acoustic signals.
